(19) United States
(12) Patent Application Publication
(10) Pub. No.: US 2005/0244365 A1
(43) Pub. Date: Nov. 3, 2005

(54) Methods, compositions, formulations, and uses of cellulose and acrylic-based polymers

(75) Inventors: Mohamed E. Labib, Princeton, NJ (US); Robert F. Rando, Annandale, NJ (US)

Correspondence Address: Rando, Robert F.;c/o Novaflux Biosciences Inc., 1 Wall Street, Princeton, NJ, 08540, US

(73) Assignee: Novaflux Biosciences, Inc., Princeton, NJ (US)

(21) Appl. No.: 10/837,153

(22) Filed: May 3, 2004

(51) Int. Cl. C08G 63/48; A61K 31/74

(52) U.S. Cl. 424/78.18; 525/54.2

(57) ABSTRACT

Compositions, formulations, and methods for the treatment or prevention, or decreasing the frequency of transmission of a virus (such as human immunodeficiency virus type 1 (HIV-1), Herpes Simplex virus type 1 (HSV1), or Herpes Simplex Virus Type 2 (HSV2), or other virus), or a bacterial infection (such as Trichomonas vaginalis, Neisseris gonorrhoeae Haemopholus ducreyl, or Chlamydia trachomatis, or other bacterial species), or a fungal infection, using an anionic cellulose- or acrylic-based oligomer, polymer, or copolymer. The present invention also includes administering a therapeutically effective amount of said oligomer, polymer, or copolymer, or a pharmaceutically acceptable salt thereof, or with a pharmaceutically acceptable carrier or diluent, thereof. The invention relies on the unique biochemical substitution of the cellulose or acrylic backbone such that the resultant molecule can remain molecularly dispersed in solution (or gel or other formulation) and mostly dissociated over a wide range of physiological microenvironments, such as the low pH found within the vaginal lumen, preferably from a pH of 14 to below 3.5. These specific substitutions also impart on the resultant molecule potent antiviral, anti-bacterial, and anti-fungal properties. In addition, these compositions can be used as general disinfectants for human use such as in contact lens solutions, mouthwashes, toothpastes, suppositories, or as more generalized disinfectants found in soaps, household cleaning products, paints, water treatments modalities, or can be incorporated into cosmetic, and can be used as vehicles for drug delivery, an adjuvant in a therapeutic formulation, or as a preservative. These compounds can be delivered in a liquid or solid dosage form and can be incorporated into barrier devices such as condoms, diaphragms, or cervical caps, to help prevent the transmission of STDs. The compounds of this invention can also be used in combination therapies with other classes of antiviral, antibacterial, or antifungal agent having similar or differing mechanisms of action including, but not limited to, anionic or cationic polymers, copolymers, or oligomers, surfactants, protease inhibitors, DNA or RNA polymerase inhibitors (including reverse transcriptase inhibitors), fusion inhibitors, cell wall biosynthesis inhibitors, integrase inhibitors, or virus or bacterial attachment inhibitors.

### FIELD OF THE INVENTION

The present invention relates to cellulose and acrylic-based polymers and uses thereof including but not limited to a method for the treatment or prevention of the transmission of infectious diseases using pharmaceutically acceptable formulations of these compounds, a method for use as a vehicle or adjuvant for use in therapeutic and cosmetic applications, a method for use as a thickener for topically administered therapeutic formulations, and a method for use as a general disinfecting agent.

Prior Art.
U.S. Pat. Nos:

| | | |
|---|---|---|
| 3,429,963 | 2/1969 | Shedlovsky, L. |
| 3,870,702 | x/1975 | Koyanagi, S. et al. |
| 3,956,480 | 5/1976 | Dichter; et al. |
| 4,138,477 | 2/1979 | Gaffar; M. C, S. |
| 4,183,914 | 1/1980 | Gaffar and Gaffar |
| 4,330,338 | 5/1982 | Banker |
| 4,385,078 | 5/1983 | Onda et al. |
| 4,462,839 | 7/1984 | McGinley et al. |
| 4,518,433 | 5/1985 | McGinley et al. |
| 4,894,220 | 1/1990 | Nabi and Gaffar |
| 4,960,814 | 10/1990 | Wu et al. |
| 4,968,350 | 11/1990 | Bindschaedler et al. |
| 5,334,375 | 8/1994 | Nabi et al. |
| 6,165,493 | 12/2000 | Neurath |
| 6,258,799 | 7/2001 | Kokubo and Nishiyama |
| 6,462,030 | 10/2002 | Neurath |

Other Publications:

Hoshi, N., Kokubo, H., Nagai, T., Obara, S. “Application of HPMC and HPMCAS to film coating of pharmaceutical dosage forms in aqueous polymeric coatings for pharmaceutical dosage forms,” 2^{nd} ed, ed. By McGinty, J. W., Marcel Decker, Inc., New York and Basel, 1997, pp. 177-225

Neurath, A. R., Strick, N., Jiang, S., Li, Y. Y., and Debnath, A. K. “Anti-HIV-1 activity of cellulose acetate phthalate: Synergy with soluble CD4 and induction of “dead-end” gp41 six-helix bundles”, BMC Infectious Diseases 2:6 (2002)

Neurath, A. R., Strick, N., Li, Y. Y., and Jiang, S., “Design of a ‘microbicide” for prevention of sexually transmitted diseases using “inactive” pharmaceutical excipients”, Biologicals 27:11-21 (1999)

Gyotoku, T., Aurelian, L., and Neurath, A. R. “Cellulose acetate phthalate (CAP): an ‘inactive’ pharmaceutical excipient with antiviral activity in the mouse model of genital herpesvirus infecton”, Antiviral Chem. Chemother 10:327-332 (1999)

Neurath, A. R., Li, Y. Y., Mandeville, R., and Richard, L., “In vitro activity of a cellulose acetate phthalate topical cream against organisms associated with bacterial vaginosis”, J. Antimicrobial Chemother. 45:713-714 (2000)

Neurath, A. R. “Microbicide for prevention of sexually transmitted diseases using a pharmaceutical excipient”, AIDS Patient Care STDS 14:215-219 (2000)

Manson, K. H. Wyand, M. S., Miller, C., and Neurath, A. R. “The effect of a cellulose acetate phthalate topical cream on vaginal transmission of simian immunodeficiency virus in rhesus monkeys”. Antimicrob. Agents Chemother 44:3199-3202 (2000)

Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120”, BMC Infectious Diseases 1:17 (2001)

Kukubo, H. Obara, S., Minemura, K., and Tanaka, T., “Development of Cellulose derivatives as novael enteric coating agents soluble at pH 3.5-4.5 and higher”, Chem Pharm. Bull. 45:1350-1353 (1997)

Maekawa, H., Takagishi, Y., Iwamoto, K., Doi, Y., and Ogura, T. “Cephalexin preparation with prolonged activity”,. Jpn J. Antibiot. 30:631-638 (1977);

Lappas, L. C., and McKeeham, W., “Polymeric pharmaceutical coating materials. II. In vivo evaluation as enteric coatings”, J. Pharm. Sci., 56:1257-261 (1967)

### BACKGROUND OF THE INVENTION

1. Field

The present invention relates to methods, compositions, and/or formulations of cellulose and acrylic-based polymers and uses thereof including, but not limited to, a method for the treatment or prevention of the transmission of infectious diseases using pharmaceutically acceptable formulations of these compounds, a vehicle or adjuvant for use in therapeutic and cosmetic applications, a thickener for topically administered therapeutic formulations, and as a disinfecting agent. This invention also covers methods, compositions, and/or formulations for treating or decreasing the frequency of transmission of sexually transmitted diseases such as, but not limited to, human immunodeficiency virus type 1, herpesviruses, Trichomonas vaginalis, Neisseris gonorrhoeae Haemopholus ducreyl, or Chlamydia trachomatis or Candida albicans, by administering topically a specifically substituted cellulose or acrylic-based polymer or oligomer such that the resultant molecule remains molecularly dispersed and mostly dissociated in aqueous solution over a wide range in pH (from 14 to below 3.5). The compounds of this invention can also be used in combination therapies with other classes of antiviral, antibacterial, or antifungal agent having similar or differing mechanisms of action including, but not limited to, anionic or cationic polymers or oligomers, surfactants, protease inhibitors, DNA or RNA polymerase inhibitors (including reverse transcriptase inhibitors), fusion inhibitors, cell wall biosynthesis inhibitors, integrase inhibitors, or virus or bacterial attachment inhibitors

2. Background Information

a. Topical Treatment to Help Prevent the Spread of Sexually Transmitted Diseases (STDs).

STDs are diseases caused by organisms that have the ability to infect tissues of, or to pass through, the anogenital tract, the oral or nasopharyngeal cavity, and have the capability of, but are not limited to, spreading between individuals via sexual contact, or poor hygiene.

Human immunodeficiency virus type 1 (HIV-1), a member of the retrovirus family, is the causative agent in the development of acquired immune deficiency syndrome (AIDS). The usual method for the spread of this virus is via sexual contact, thus the classification of HIV-1 as a STD (Mann, J., M., Tarantola, D. J. M., Netter, T. W., “AIDS in the World”, Cambridge: Harvard University Press, (1992)). The AIDS condition is a catastrophic, fatal disease that presently infects millions of people worldwide. Major efforts are being made to develop novel antiviral agents with unique mechanisms of action to be used in drug therapy and methods of preventing the transmission of HIV-1, methods of curing the AIDS disease state once contracted, and methods of ameliorating the symptoms of AIDS.

The spread of HIV-1 has been postulated to be facilitated by prior infection with other STD pathogens (Perine, P. L. “Sexually Transmitted Disease in the Tropics”, Med. J. Aust. 160:358-366 (1994)). Therefore one strategy for combating the spread of HIV-1 that has proven to be economically justifiable is via the treatment of STDs other than HIV (St. Louis, M. E., et al., “HIV prevention through early detection and treatment of other sexually transmitted diseases-United States recommendation of the advisory committee for HIV and STD prevention”, Morb. Mort. Wkly. Rep. 47 (RR-12), 1-24 (1998); Over, M. and Piot, P. “Human Immunodeficiency Virus Infection and Other Sexually Transmitted Diseases in Developing Countries: Public Health Importance and Priorities for Resource Allocations”, J. Infect. Dis. 174 (suppl. 2) 162-175 (1996)). The indicated pathogens include, but are not limited to other viral infections like HSV2, or one of the many anogential human papillomavirus genotypes (HPV), bacterial infections including Trichomonas vaginalis, Neisseris gonorrhea Haemopholus ducreyl, or Chlamydia trachomatis, and yeast infections such as Candida albicans.

In the absence of prophylactic vaccines against most of the indicated STDs, and lack of safe anti-infective agents that are affordable in developing countries, other simple methods to control the transmission of STDs, including HIV-1, must be sought. This includes mechanical (condom) and chemical barrier methods (microbicides) or combinations thereof. A microbicide is a chemical entity that can prevent or reduce transmission of sexually-transmitted infections when applied to the vagina or rectum

Formulations of spermicides shown in vitro to inactivate STD pathogens have been considered for use in this regard, but based upon clinical safety and efficacy trials undertaken to date, the need for newer, novel agents is still evident. For example, vaginal contraceptive products have been available for many years and usually contain nonoxynol-9 (N-9) or other detergent/surfactant as the active ingredient. These products have an inherent toxicity to the vaginal and cervical tissues. Therefore frequent use of N-9 causes irritation and inflammation of the vagina (M. K. Stafford et al “Safety study of nonoxynol-9 as a vaginal microbicide: evidence of adverse effects”, J. AIDS Human Retrovirology, 17:327-331 (1998)). N-9 is also known to activate the local immune response and potentiate the transport of immune cells to the mucosal surface leading to increase in the potential for virus infection (Stevenson, J. “Widely used spermicide may increase, not decrease, risk of HIV transmission” JAMA 284:949, (2000)). N-9 is also toxic to vaginal and cervical cells increasing the permeability of vaginal tissue, and can inactivate lactobacilli. Lactobacilli produce lactic acid and hydrogen peroxide that serve to maintain the acidic pH of the vagina (˜pH 3.5 to 5.0). At this pH a number of STD causing organisms as well as spermatozoa are inactivated to a degree. Disturbance of the vaginal microbial flora can lead to vaginal infections, which in turn increase the chance of HIV/STD transmission.

For these reasons a set of criteria can be put forth to help define the qualities that will lead to a microbicide candidate with a good chance of successfully reaching commercialization. For example, an anti-viral microbicide should (i) be effective against infection caused by cell-free and cell-associated virus, (ii) adsorbs tightly with its molecular target(s), i.e., its adsorption should not be reversed by dilution or washing, (iii) permanently “inactivate” the virus, (iv) inactivate free virus and infected cells faster than their rate of transport through the mucus layer, (v) have persistent activity for more than one episode of coitus, (vi) be safe to host cells and tissues—causing no irritation or lesions, (vii) be effective over a wide range of pH found in the vaginal lumen before, during and post-coitus, (viii) be easy to formulate, (ix) remain stable in the formulated state, (x) not activate mucosal immunity, (xi) retard transport in mucus and entire vaginal and rectal mucosa, and (xii) be inexpensive for worldwide application. It is unlikely that one candidate microbicide can fulfill all of these criteria, but it is put forward to demonstrate the difficulties one may encounter in the discovery and development of an effective anti-STD agent. As with systemic anti-HIV treatment regimens, combination therapy will undoubtedly enhance the overall performance of any STD therapeutic regimen. The compounds described in this application can be used in combination with other classes of antiviral, antibacterial, or antifungal agent having similar r differing mechanisms of action including, but not limited to, anionic or cationic polymers or oligomers, surfactants, protease inhibitors, DNA or RNA polymerase inhibitors (including reverse transcriptase inhibitors), fusion inhibitors, cell wall biosynthesis inhibitors, integrase inhibitors, or virus or bacterial attachment inhibitors

Many of the compounds that are under evaluation or have been previously evaluated as HIV-1 microbicide candidates meet some of the above listed criteria and usually fall into two categories—either surfactants or polyanionic polymers (Pauwels, R., and De Clercq, E. “Development of vaginal microbicides for the prevention of heterosexual transmission of HIV”, J. AIDS Hum Retroviruses 11:211-221 (1996); “Recommendations for the development of vaginal microbicides”, International Working Group on Vaginal Microbicides AIDS 10:1-6 (1996)). However, these aforementioned agents may not satisfy enough of the proposed criteria for a successful microbicide as mentioned above. In addition, most of the compounds under current investigations as microbicides are non-specific and emerged from either pharmaceutical excipients or compounds used in conventional topical formulations—almost none of the compounds used have definite chemical formulae, and many are based on natural or synthetic water-soluble polymers. For example, despite the effectiveness of N-9 with respect to HIV-1 inactivation in vitro, its failure to effectively prevent HIV-1 infection in vivo has been attributed to its high irritation profile and indiscriminate disruption of epithelial cells (Feldblum, P. J., and Rosenberg, M. J., “Spermicides and sexually transmitted diseases: new perspectives.” N.C. Med J. 47:569-572 (1986); Alexander, N.J., “Sexual transmission of human immunodeficiency virus: virus entry into the male and female genital tract”, WHO Global Programme on AIDS Fertil Steril. 54:1-18 (1990); Niruthisard, S., Roddy, R. E., and Chutivongse, S, “The effects of frequent nonoxynol-9 use on the vaginal and cervical mucosa.” Sex Transm Dis 18:176-179 (1991); Roddy, R. E., et al. “A dosing study of nonoxynol-9 and genital irritation.”, J STD AIDS 4:165-170 (1993); Kreiss et al. “Efficacy of nonoxynol 9 contraceptive sponge use in preventing heterosexual acquisition of HIV in Nairobi prostitutes.” JAMA 268:477-482 (1992); Catalone, B. J., et al. “Mouse model of cervicovaginal toxicity and inflammation for the preclinical evaluation of topical vaginal microbicides.” Antimicrobial Agents and Chemotherapy in press (2004)). In order to satisfy the diverse criteria stated above, the target molecule needs to be custom tailored to provide several functions at the same time. Unfortunately, the ability to manipulate, by synthetic means, the molecular structure of the current classes of agents (e.g. surfactants such as N-9 or C31G, or sulfated polysaccharides) is limited, or in some cases even impossible.

Therefore it is extremely important to identify and evaluate new antimicrobial agents which can be used vaginally in effective doses or formulations without inactivating lactobacilli or causing overt vaginal irritation or other toxicity.

Recent work conducted at the New York Blood Center has focused on the use of two promising anionic polymers, cellulose acetate phthalate (CAP) and hydroxypropyl methylcellulose phthalate (HPMCP). Both of these polymers have demonstrated excellent activity against a wide range of sexually transmitted organisms including HIV-1 (Neurath et al. “Methods and compositions for decreasing the frequency of HIV, Herpesvirus and sexually transmitted bacterial infections.” U.S. Pat. No. 6,165,493, (2000); Neurath, A. R. “Method for inactivating bacteria associated with bacterial vaginosis using cellulose acetate phthalate and /or hydroxypropyl methycellulose phthalate.” U.S. Pat. No. 6,462,030 (2002); Neurath, A. R., et al. “Anti-HIV-1 activity of cellulose acetate phthalate: Synergy with soluble CD4 and induction of “dead-end” gp41 six-helix bundles.” BMC Infectious Diseases 2:6 (2002); Neurath, A. R., Strick, N., Li, Y. Y., and Jiang, S., “Design of a “microbicide” for prevention of sexually transmitted diseases using “inactive” pharmaceutical excipients.” Biologicals 27:11-21 (1999); Gyotoku, T., Aurelian, L., and Neurath, A. R. “Cellulose acetate phthalate (CAP): an ‘inactive’ pharmaceutical excipient with antiviral activity in the mouse model of genital herpesvirus infecton.” Antiviral Chem. Chemother 10:327-332 (1999); Neurath, A. R., Li, Y. Y., Mandeville, R., and Richard, L., “In vitro activity of a cellulose acetate phthalate topical cream against organisms associated with bacterial vaginosis.” J. Antimicrobial Chemother. 45:713-714 (2000); Neurath, A. R. “Microbicide for prevention of sexually transmitted diseases using a pharmaceutical excipients.” AIDS Patient Care STDS 14:215-219 (2000); Manson, K. H. Wyand, M. S., Miller, C., and Neurath, A. R. “The effect of a cellulose acetate phthalate topical cream on vaginal transmission of simian immunodeficiency virus in rhesus monkeys.” Antimicrob. Agents Chemother 44:3199-3202 (2000); Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120.” BMC Infectious Diseases 1:17 (2001)).

CAP and HPMCP were first developed for use as pharmaceutical excipients in enteric coating to help protect pharmaceutical preparations from degradation by the low pH of gastric juices, and to simultaneously protect the gastric mucosa from irritation by the drug. One desirable attribute of these coatings was the ability to not dissolve until the drug substance reached the intestines where the pH is mostly neutral or alkaline. There is a large difference in pH between the stomach and the intestines. In the stomach gastric juice pH values range from 1.5 to 3.5 while in the intestines the pH values are much higher ranging from 3.6 to 7.9. The pH in the duodenum closest to the stomach has a lower pH due to the transfer of material from the stomach to the intestines, however at the point of nutrient uptake by the intestines the pH has moved into the neutral or slightly alkaline range (“Remington's Pharmaceutical Sciences,” 14^{th} ed., Mack Publishing Co., Easton, Pa., 1970, p. 1689-1691; Wagner, J. G., Ryan, G. W., Kubiak, E., and Long, S., “Enteric Coatings V. pH Dependence and Stability”, J. Am. Pharm. Assoc. Sci., 49:133-139, (1960); Kokubo, H., et al., “Development of Cellulose derivatives as novel enteric coating agents soluble at pH 3.5-4.5 and higher”, Chem. Pharm. Bull 45:1350-1353 (1997)). Commercially available enteric coating agents of both cellulosic and acrylic polymers are soluble in the pH range from 5.0 to 7.0 (Kokubo, H., et al., “Development of Cellulose derivatives as novel enteric coating agents soluble at pH 3.5-4.5 and higher.” Chem. Pharm. Bull 45:1350-1353 (1997); Maekawa, H., Takagishi, Y., Iwamoto, K., Doi, Y., and Ogura,T. “Cephalexin preparation with prolonged activity.” Jpn J. Antibiot. 30:631-638 (1977); Lappas, L. C., and McKeeham, W., “Polymeric pharmaceutical coating materials. II. In vivo evaluation as enteric coatings.” J. Pharm. Sci., 56:1257-261 (1967); Hoshi, N., Kokubo, H., Nagai, T., Obara, S. “Application of HPMC and HPMCAS to film coating of pharmaceutical dosage forms in aqueous polymeric coatings for pharmaceutical dosage forms.” 2^{nd} ed, ed. By McGinty, J. W., Marcel Decker, Inc., New York and Basel, 1997, pp. 177-225), however, in drugs with poor and limited absorbability in the gastro-intestinal tract it is desirable to ensure that the coating is dissolved as early as possible by reducing the dissolution pH thereof, in order to maximize the drug absorption. This problem in solubility at low pH (3.5 to 5.5) has been found to be the case for both CAP and HPMCP. CAP and HPMCP are insoluble in aqueous solutions unless the pH is ˜6.0 or above (Neurath A. R. et al. “Methods and compositions for decreasing the frequency of HIV, Herpesvirus and sexually transmitted bacterial infections.” U.S. Pat. No. 6,165,493 (2000)).

This differential in pH solubility is extremely important for agents that have potential use as inhibitors of sexually transmitted diseases. Vaginal secretions from healthy, reproductive-age women, are usually acidic with pH values in the range of 3.4 to 6.0 (S. Voeller, D. J. Anderson, “Heterosexual Transmission of HIV” JAMA 267, 1917-1918 (2000)). The pH of the vaginal lumen may then fluctuate transiently upon the addition of semen. Consequently the topical application of a formulation in which either CAP or HPMCP would be soluble (i.e. pH ˜6.0) would be expected to precipitate out of solution once they come in contact with the “acidic” vaginal environment. Furthermore the dissolution time is sufficiently long for this class of compound which indicates that the active agent may not have time to regain solubility post-coitus when the pH has been transiently raised (Kokubo, H., et al., “Development of Cellulose derivatives as novel enteric coating agents soluble at pH 3.5-4.5 and higher”, Chem. Pharm. Bul.l 45:1350-1353 (1997). Moreover, if the polyanionic electrostatic nature of the molecules is diminished due to lack of dissociation of the molecule's carboxyl group in the vagina, the protective property of the molecule is expected to decrease or even disappear completely. It is therefore of interest from both a pharmaceutical coating point of view and from a putative topical microbicide perspective that polymers soluble at more acidic pH than conventional enteric coatings are designed and tested for biological, or pharmacological benefit.

As stated above the original utility of CAP and HPMCP was with respect to enteric coating. Another class of molecules widely used in pharmaceutical applications for their excellent film-forming properties and high quality bio-adhesive performance is acrylic co-polymers that also contain a periodic carboxylic acid group. Gantrez (Gantrez® International Specialty Products or ISP) is one such co-polymer made from the polymerization of methylvinyl ether and maleic anhydride (poly methyl vinyl ether/maleic anhydride (MVE/MA)). MVE/MA and similar agents are used as thickeners, complexing agents, denture adhesive base, buccal/transmucosal tablets, transdermal patches (Degim, I. T., Acarturk, F, Erdogan, D., and Demirez-Lortlar, N. “Transdermal administration of bromocriptine.” Biol. Pharm. Bull. 26:501-505, (2003)), topical carriers or micro spheres for mucosal delivery of drugs (Kockisch, S., Rees, G. D., Young, S. A., Tsibouklis, J., and Smart, J. D. “Polymeric microspheres for drug delivery to the oral cavity: an in vitro evaluation of mucoadhsive potential.” J. Pharm. Sci. 92:1614-1623, (2003); Foss, A. C., Goto, T., Morishita, M., and Peppas, N. A., “Development of acrylic-based copolymers for oral insulin delivery.” Eur. J., Pharm. Biopharm. 57:163-169, (2004)), enteric film coating agents, wound dressing applications (Tanodekaew, S., Prasitsilp, M., Swasdison, S., Thavornyutikarn, B., Pothsree, T., and Pateepasen, R. “Preparation of acrylic grafted chitin for would dressing application.” Biomaterials :1453-1460, (2004)), and hydrophilic colloids. One form of Gantrez is mixed with triclosan in toothpaste with claims of extended control of breath odor for over 12 hours (Sharma, N. C., Galustians, H. J., Qaquish, J., Galustians, A., Rustogi, K. N., Petrone, M. E., Chanknis, P. Garcia, L., Volpe, A. R., and Proskin H. M., “The clinical effectiveness of dentifrice containing triclosan and a copolymer for controlling breath odor measured organoleptically twelve hours after tooth brushing.” J. Clin. Dent. 10:1310134, (1999); Zambon, J. J., Reynolds, H. S., Dunford, R. G., and Bonta, C. Y., “Effect of triclosan/copolymer/fluoride dentifrice on the oral microflora.” Am. J. Dent. 3S27-34, (1990)). Certain acrylic-based copolymers are also being studied for use in diagnosis of cancer (Manivasager, V., Heng, P. W., Hao, J., Zheng, W., Soo, K. C., and Olivo, M. “A study of 5-aminolevulinic acid and its methyl ester used in in vitro and in in vivo system so human bladder cancer.” Int. J. Oncol. 22:313-318, (2003)). Maleic acid copolymers with methyl vinyl ether are also being used in model systems to covalently immobilize peptides and other macromolecules via the formation of amide bonds (Ladaviere, C., Lorenzo, C., Elaissari, A., Mandrand, B., and Delair, T. “Electrostatically driven immobilization of peptides onto (Maleic anhydride-alt-methyl vinyl ether) copolymers in aqueous media.” Bioconj. Chem. 11:146-152, (2000)). Divinyl ether and maleic anhydride copolymers have been used to retard the development of artificially induced metastases and to activate macrophages to non specifically attack tumor cells (Pavlidis, N. A., Schultz, R. M., Chirigos, M. A. and Luetzeler, J. “Effect of maleic anhydride-divinyl ether copolymers on experimental M109 metastases and macrophage tumoricidal function.” Cancer Treat Rep. 62:1817-1822, (1978)). In these studies the investigators found that the lower molecular weight polymers were most effective. This is similar to the results obtained using divinyl ether and maleic anhydride copolymers linked to derivatives of the antiviral agent adamantine (Kozeletskaia, K. N., Stotskaia, L. L., Serbin, A. V., Munshi, K., Sominina, A. A., and Kiselev, O. I. “Structure and antiviral activity of adamantine-containing polymer preparation.” Vopr VIrousol. 48:19-26, (2003)). In these experiments the adamantine containing copolymers were shown to inhibit a variety of viruses in vitro including influenza, herpes simplex type 1, and parainfluenza. The efficiency of the antiviral effect depended upon the molecular weight of the polymer (lower molecular weight was better) and the structure of the linkage between the adamantine and the copolymer. In this present application the inventors demonstrate that a copolymer of maleic acid and methyl vinyl ether without any additional derivitization is capable of inhibiting HIV-1 transmission in vitro.

b. Sexually Transmitted Viral Infections.

Despite almost 20 years of AIDS prevention efforts and research, the sexually transmitted HIV-1 and HIV-2 epidemic continues to be a major health problem throughout the world and is accelerating in many areas. To date the HIV epidemic has infected over 42 million people predominantly through sexual intercourse at the end of 2002. Of these there have been 3.1 million cumulative deaths from the disease worldwide (from the Joint United Nations Program on HIV/AIDS and the World Health Organization's AIDS Epidemic Update Report, December 2002).

HIV-1 and HIV-2 are retroviruses and share about 50% homology at the nucleotide level, contain the same complement of genes, and appear to have similar infectious cycles within human cells. The genetic material for retroviruses is Ribonucleic Acid (RNA) and encoded within their genomes are their polymerase (reverse transcriptase or RT), protease and integrase enzymes essential for the viral life cycle. The RT enzyme catalyzes synthesis of a complementary DNA strand from the viral RNA templates, the DNA helix then inserts into the host genome with the aid of the HIV integrase enzyme. The integrated DNA may persist as a latent infection characterized by little or no production of virus or helper/inducer cell death for an indefinite period of time. When the viral DNA is transcribed and translated by the infected cells, new viral RNA and proteins are produced. The viral proteins are processed into mature entities by the viral protease enzyme and these processed proteins are assembled into the structure of the mature virus particle.

Despite the remarkable advances that have been made in the last 20 years regarding the molecular virology, pathogenesis and epidemiology of HIV, the development of an effective HIV vaccine remains an elusive goal even though efforts have been ongoing in this regard since the first positive identification of HIV as the causative agent in the development of AIDS. The major reasons for the lack of success in the development of a vaccine are various including integration of the virus into the host cell genome, infections of long-lived immune cells, HIV genetic diversity (especially in its envelope), persistent high viral replication releasing up to 10 billion viral particles per day and/or production of immunosuppressive products or proteins. Despite the technical hurdles a great deal of effort using a variety of different strategies are ongoing in this area. For example, live attenuated simian immunodeficiency virus (SIV) has been shown to protect macaques (Daniel, M. et al. “Protective effects of a live attenuated SIV vaccine with a deletion in the nef.” Science 258:1938-1941 (1992)), however the use of a live attenuate HIV vaccine is unlikely due to safety concerns (Baba, T., et al., “Live attenuated, multiply defected simian immunodeficiency viruses causes AIDS in infant and adult macaques.” Nature Med. 5:194-203 (1999)). Therefore a number of recombinant viral vectors such as modified vaccinia virus Ankara, canarypox virus, measles virus, and adenovirus have been evaluated in preclinical or clinical trials (Mascola, J. R., and G. J. Nabel, “Vaccines for he prevention of HIV-1 disease.” Curr. Opin. Immunol. 13:489-495 (2001); Lorin, C., et al. “A single injection of recombinant measles virus vaccines expressing human immunodeficiency virus (HIV) type 1 Clade B envelope glycoproteins induces neutralizing antibodies and cellular immune responses to HIV.” J. VIrol. 78:146-157 (2004)). Given all of this work, at the present time and in the foreseeable future, there is no effective vaccine for HIV (either prophylactic or therapeutic).

At the same time a great deal of success has been achieved in the development of therapies and therapeutic regimens for the systemic treatment of HIV infections. Virtually all the compounds that are currently used or are the subject of advanced clinical trials for the treatment of HIV belong to one of the following classes:
1) Nucleoside analogue inhibitors of reverse transcriptase functions.
2) Non-nucleoside analogue inhibitors of reverse transcriptase functions
3) HIV-1 Protease inhibitors.
4) Virus fusion inhibitors (the 36 amino acid fusion inhibitor T20 has recently been approved for sale by the FDA).

The HIV- 1 replication cycle can be interrupted at many different points. As indicated by the approved medications, viral reverse transcriptase and protease enzymes are good molecular targets, as is the entire process by which the virus fuses to and injects itself into host cells. Thus the recently approved drug T20 (Fuzeon) is the first in a novel class of anti-HIV-1 agents. However, in addition to the drugs already approved for treatment of HIV-1 infection, work continues on the discovery and development of additional treatment modalities because of the virus's propensity to mutant and thus renders ineffective the existing therapies.

At present combination therapy comprising at least three anti-HIV drugs has become the standard treatment for HIV infected patients. Virtually all drugs that have been licensed for clinical use for the treatment of HIV infection fall into one of the four categories listed above, comprising three molecular targets. However one problem with current therapy is the cost associated with the need to use multiple drugs used in combination. Estimates of $15000 to $20000 U.S. per year per person are close approximations. This cost makes it virtually impossible for many people to afford combination therapy, especially in developing nations where the need is greatest. Another problem with existing therapeutic regimens, as stated above is the ability of the virus to develop resistance to the individual medications and many times to develop resistance to the combination therapy. This works against the population in two ways. First, the individual infected will eventually run out of treatment options and second, if the infected individual passes along a virus already resistant to many existing therapeutic agents, the newly infected individual will have a more limited treatment option than the first. Therefore, the need for new, improved and hopefully inexpensive medications to prevent the transmission of the disease (in lieu of a vaccine) is evident.

Most importantly in the search for new medications to combat the spread of the HIV is the search for chemotherapeutic interventions that work by novel mechanism(s) of action. Several potential areas for intervention that are under consideration or have active programs in include 1) blocking the viral envelope glycoprotein gp120, 2) additional mechanisms beyond gp120 to block virus entry such as blocking the virus receptor CD4 or co-receptors CXCR4 or CCR5, 3) viral assembly and disassembly through targeting the zinc finder domain of the viral nucleocapsid protein 7 (NCp7) and 4) by interfering with the functions of the viral integrase protein, and by interruption of virus specific transcription processes.

The mechanism by which HIV passes through the mucosal epithelium to infect underlying target cells, in the form of free virus or virus-infected cells, has not been fully defined. In addition, the type of cells infected by the virus could be derived from any one, or more, of a multitude of cell types (Miller, C. J. et al. “Genital Mucosal Transmission of Simian Immunodeficiency Virus: Animal Model for Heterosexual Transmission of Human Immunodeficiency Virus.” J. Virol. 63:4277-4284 (1989); Phillips, D. M. and Bourinbaiar, A. S. “Mechanism of HIV Spread from Lymphocytes to Epithelia.” Virology 186, 261-273 (1992); Philips, D. M., Tan X., Pearce-Pratt, R. and Zacharopoulos, V. R., “An Assay for HIV Infection of Cultured Human Cervix-derived Cells.” J. Viro.l Methods, 52, 1-13 (1995); Ho, J. L. et al, “Neutrophils from Human Immunodeficiency virus (HIV)-seronegative Donors Induce HIV Replication from HIV-infected patients Mononuclear Cells and Cell lines. An In Vitro Model of HIV Transmission Facilitated by Chlamydia Trachomatis.” J. Exp. Med., 181, 1493-1505 (1995); Braathen, L. R., and Mork, C., in “HIV infection of Skin Langerhans Cells”, In: Skin Langerhans (dendritic) cells in virus infections and AIDS (ed Becker, Y.) 131-139, Kluwer Academic Publishers, Boston, (1991). Such cells include T lymphocytes, monocytes/macrophages and dendritic cells, suggesting that CD4 cell receptors are engaged in the process of virus transmission as is well documented for HIV infection in blood or lymphatic tissues (Parr M. B., and Parr E. L., “Langerhans Cells and T lymphocytes Subsets in the Murine Vagina and Cervix.” Biology and Reproduction 44, 491-498 (1991); Pope, M. et al. “Conjugates of Dendritic Cells and Memory T Lymphocytes from Skin Facilitate Productive Infection With HIV-1.” Cell 78, 389-398 (1994); and Wira, C. R. and Rossoll, R. M. “Antigen-presenting Cells in the Female Reproductive Tract: Influence of Sex Hormones on Antigen Presentation in the Vagina.” Immunology, 84, 505-508 (1995)).

Herpes viruses are large double stranded DNA viruses, with genome sizes usually greater than 120,000 base pairs (for review see “Herpesviridae; A Brief Introduction”, Virology, Second Edition, edited by B. N. Fields, Chapter 64, 1787 (1990)). HSV1 is one of the most common infections in the U.S. with infection rates estimated to be greater than 50% of the population. All herpes virus types encode their own polymerase and many times their own thymidine kinase. For this reason most of the approved antiviral agents target the DNA polymerase enzyme of the virus and/or use the viral thymidine kinase for conversion from prodrug to active agent thereby gaining specificity for the infected cell. Herpes viruses are another class of virus that like HIV-1 develop resistance to existing therapy, and can cause problems from a STD as well as a chronic infection point of view. For example human cytomegalovirus (HCMV) is a serious, life threatening opportunistic pathogen in immuno-compromised individuals such as AIDS patients (Macher, A. M., et al., “Death in the AIDS patients: role of cytomegalovirus.” NEJM 309:1454 (1983); Tyms, A. S., Taylor, D. L., and Parkin, J. M., “Cytomegalovirus and the aquired immune deficiency syndrome.” J Anitmicrob Chemother 23 SupplementA:89-105 (1989)) or organ transplant recipients (Meyers, J. D., “Prevention and treatment of cytomegalovirus infections.” Annual Rev. Med. 42:179-187 (1991)). Over the past decade there has been a tremendous effort dedicated to improving the available treatments for herpes viruses. At the present time acyclovir is still the most prescribed drug for HSV1 and HSV2, while for HCMV ganciclovir, foscarnet, cidofovir, and fomivirsen are the only drugs currently available (Bédard et al., “Antiviral properties of a series of 1,6-naphthyridine and dihydroisoquinoline derivatives exhibiting potent activity against human cytomegalovirus.” Antimicrob. Agents and Chemother. 44:929-937 (2000)). However, none of these systemic treatments are effective at preventing the sexual transmission of viruses; therefore there is still an urgent need for new drugs that have unique mechanisms of action and modes of therapeutic intervention.

Members of the Herpes virus family that infect humans (in Herpesviridae; A Brief Introduction”, Virology, Second Edition, edited by B. N. Fields, Chapter 64, 1787 (1990)) and disease(s) with which they are commonly associated include:

Herpes Simplex Virus Type 1 (HSV1) is a recurrent viral infection characterized by the appearance on the cutaneous or mucosal surface membranes of single or multiple clusters of small vesicle, filled with clear fluid on a slightly raised inflamed base (herpes labialis). In addition, gingivostomatitis may occur as a result of HSV1 infection in infants (Kleymann, G., “New antiviral drugs that target herpesvirus helicase primase enzyme.” Herpes 10:46-52 (2003); in Herpesviridae; A Brief Introduction”, Virology, Second Edition, edited by B. N. Fields, Chapter 64, 1787 (1990)).

Herpes Simplex Virus Type 2 (HSV2) causes genital herpes and vulvovaginits may occur as a result of HSV2 infection in infants (Kleymann, G., “New antiviral drugs that target herpesvirus helicase primase enzyme.” Herpes 10:46-52 (2003)).

Human Cytomegalovirus (HCMV) infections are a common cause of morbidity and mortality in solid organ and haematopoietic stem cell transplant recipients (Razonable, R. R., and Paya, C. V., “Herpesvirus infections in transplant recipients: current challenges in the clinical management of cytomegalovirus and Epstein-Barr virus infections.” Herpes 10:60-65 (2003)).

Varicella-Zoster Virus (VZV) causes varicella (chickenpox) and Zoster (shingles) (Vazquez, M., “Varicella Zoster virus infections in children after introduction of live attenuated varicella vaccine.” Curr. Opin. Pediatr. 16:80-84 (2004)).

Epstein—Barr virus (EBV) is the causative agent of infectious mononucleosis and has been associated with Burkett's lymphoma and nasopharyngeal carcinoma,

Human Herpesvirus 6 (HHV6) is a very common childhood disease causing exanthem subitum (roseola) (Boutolleau, D., et al., “Human herpesvirus (HHV)-6 and HHV-7; two closely related viruses with different infection profiles in stem cell transplant recipients”, J. Inf. Dis. (2003)).

Herpes Simplex Virus Type 7 (HSV7) is a T-lymphotropic herpesvirus and can cause exanthem subitum. Pathogenesis and sequelae of HH7 however are poorly understood (Dewhurst, S., Skrincosky, D., and van Loon, N. “Human Herpesvirus 7”, Expert Rev Mol. Med. 18:1-10(1997)).

Herpes Simplex Virus Type 8 (HSV8). The molecular genetics of the human herpesvirus 8 (HHV8) has now been characterized and the virus appears to be important in the pathogenesis of Kaposi's sarcoma (KS) (Hong, a, Davies, S. and Lee, S. C., “Immunohistochemical detection of the human herpesvirus 8 (HHV8) latent nuclear antigen-1 in Kaposi's sarcoma.” Pathology 35:448-450 (2003); Cathomas, G., “Kaposi's sarcoma-associated herpesvirus (KSHV)/human herpsevirus 8 (HHV8) as a tumor virus.” Herpes 10:72-77 (2003)).

In addition to infections in humans, herpes viruses have also been isolated from a variety of animals and fish (in “Herpesviridae; A Brief Introduction.” Virology, Second Edition, edited by B. N. Fields, Chapter 64, 1787 (1990)).

While HSV1 infections are more common than HSV2 it is still estimated that up to 20% of the U.S. population are infected with HSV2. HSV2 is associated with the anogenital tract, is sexually transmitted, causes recurrent genital ulcers, and can be extremely dangerous to newborns (causing viremia or even a fatal encephalitis) if transmitted during the birthing process (Fleming, D. T., McQuillan, G. M. Johnson, R. E. et al. “Herpes simplex virus type 2 in the Unites Sates, 1976 to 1994.” N. Eng. J. Med 337:1105-1111 (1997); Arvin, A. M., and Prober, C. G., “Herpes Simplex Virus Type 2—A Persistent Problem.” N. Engl. J. Med. 337:1158-1159 (1997)). Although, as stated above, there are treatments available for HSV1 and HSV2, efficacious long-term suppression of genital herpes is expensive (Engel, J. P. “Long-term Suppression of Genital Herpes.” JAMA, 280:928-929 (1998)). The probability of further spread of the virus by untreated people and asymptomatic carriers not receiving antiviral therapy is extremely high considering the high prevalence of the infections. It is thought that other herpesviruses including HCMV (Krieger, J. M., Coombs, R. W., Collier, A. C. et al. “Seminal Shedding of Human Immnodeficiency virus Type 1 and Human Cytomegalovirus: Evidence for Different Immunologic Controls.” J. Infect. Dis. 171:1018-1022 (1995); van der Meer, J. T. M., Drew, W. L., Bowden, R. A. et al. “Summary of the International Consensus Symposium on Advances in the Diagnosis, Treatment and Prophylaxis of Cytomegalovirus Infection.” Antiviral Res. 32:119-140 (1996)), herpesvirus type 6 (Leach, C. T., Newton, E. R., McParlin, S. et al. “Human Herpesvirus 6 Infection of the female genital tract.” J. Infect. Dis. 169:1281-1283 (1994)), and herpesvirus type 8 (Howard, M. R., Whitby, D., Bahadur, G. et al. “Detection of Human Herpesvirus 8 DNA in Semen from HIV-infected Individuals but Not Healthy Semen Donors.” AIDS 11:F15-F19 (1997)) are also transmitted sexually.

Vaccine development for herpes viruses has met with limited success. A vaccine based on the OKA strain of varicella zoster virus is commercially available, but to date no therapeutic or prophylactic herpes vaccinations that can treat or stop the spread of other herpes diseases are available (Kleymann, G., “New antiviral drugs that target herpesvirus helicase primase enzymes.” Herpes 10:46-52 (2003)). At the present time there are several ongoing efforts to develop effective vaccines against HSV1 and HSV2, most of which focus on key glycoproteins on the viral envelope (for example, Jones, C. A., and Cunningham, A. L., “Development of prophylactic vaccines for genital and neonatal herpes.” Expert Rev. Vaccines 2:541-549 (2003); Cui, F. D., et al., “Intravascular naked DNA vaccine encoding glycoprotein B induces protective humoral and cellular immunity against herpes simplex virus type 1 infection in mice.” Gene Therapy 10:2059-2066 (2003)). Therefore, as in the case of HIV, at this time there is an urgent need for inexpensive antiviral compounds that can be applied topically to help decrease the frequency of transmission of various members of the herpes virus family.

b. Sexually Transmitted Bacterial Infections.

Sexually transmitted infections of bacterial origin are among the most common infectious diseases in the United States and throughout the world. In the U.S. alone there were conservative estimates of over 4 million new cases in 1996 of three major bacterial infections, namely syphilis, gonorrhea (Neisseria gonorrhea), and Chlamydia (U.S. Government, National Institutes of Health, National Institutes of Allergy and Infectious Disease web site (factsheets/stdinfo)). Even this large number of infections is under estimating the true prevalence of these diseases. The dramatic under reporting of STDs is due to the reluctance of infected individuals to discuss their sexual health issues. In fact it has been estimated that in addition to the approximate 600,000 cases of Chlamydia reported in 1999, an additional 3 million unreported cases occurred (U.S. Government, Center for Disease Control and Prevention, National Center for HIV, STD, and TB Prevention, Division of Sexually Transmitted Diseases web site (nchstp/dstd)). In addition, worldwide there is over a 300 million annual incidence of bacterial STDs (Gerbase, A. C., Rowley, J. T., Heymann, D. H. L., et al. “Global prevalence and incidence estimates of selected curable STDs.” Sex. Transm. Inf. 74 (suppl. 1): S12-S16 (1998)). While many types of bacterial infections are treatable with antibiotics which can be relatively inexpensive (compared to most antiviral agents) if they are off patent, even the once easily cured gonorrhea has become resistant to many of the older, traditional, antibiotics. For this reason alone newer drugs that treat, prevent or decrease the transmission rate for sexually transmitted bacteria are urgently needed.

### SUMMARY OF THE INVENTION

The present invention includes compounds of Formulas I and II, their mixtures, and pharmaceutically acceptable salts or therapeutic formulations thereof, including combination therapy with one or more anti-infective drug or agent. Formula I contains a cellulosic-based polymer substituted at R.

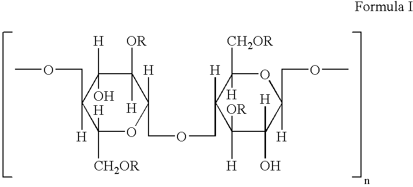

Wherein: substitution(s) at R are organic in nature and can be homogeneous or heterogeneous. The substituting group(s) can be derived from but are not limited to the examples put forth below and in Table 1 such that at least one substituted position R has a moiety containing an anionic functional group resulting in a molecule that will be molecularly dispersed and mostly dissociated over a wide range of pH from 14 to 3.5 or below. R can be derived from one or more of the following, alone or as mixed additions to the backbone, —H, —CH₃, or —CH₂CH(OH)CH₃, or acetic acid, or any monocarboxylic acid combined with moieties derived from trimellitic acid, or hydroypropyl trimellitic acid as shown below. Alternatively, R can be derived from any multi-carboxylic acid as shown in (but not limited to) Table 1 such that the upon covalent addition to the cellulose or acrylic polymer backbone the resultant R moiety has one or more carboxylic acid group remaining free and the entire molecule has the ability to remain molecularly dispersed and mostly dissociated in aqueous solutions over a wide pH range (e.g. from below 3.5 to 14). An aromatic or aliphatic organic R moiety can contain a carboxyl, sulfate or sulfonate group such that upon covalent addition to the cellulose or acrylic polymer backbone the resultant molecule has one or more carboxylic acid, sulfate or sulfonate groups exposed to the solvent environment and the entire molecule has the ability to remain molecularly dispersed and mostly dissociated in aqueous solutions over a wide pH range (e.g. from below 3.5 to 14). The carboxylic, sulfonic or sulfate acid containing moieties can be covalently attached to the polymer or oligomer backbone via several different mechanisms that one skilled in the art will appreciate including an ester or ether linkage scheme using an anhydride or acid chloride intermediate. Therefore, any solvent exposed anionic functional group added to the cellulose backbone through position R is attached to the cellulose backbone through an organic linker.

Fore example:

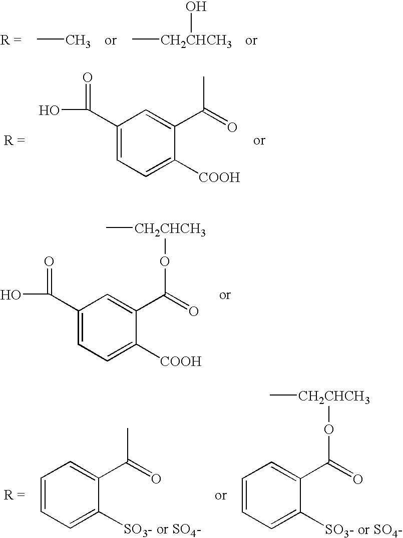

The carboxylic acid, sulfonate, or sulfate moieties on the phenol ring, or any aromatic system used, in the examples shown may be found at various, and more than one, positions.

It is also possible to further substitute a molecule described in Formula I at one or more free hydroxyl groups with an anionic agent such as a sulfate or sulfonate group such that the resultant molecule has an enhanced electrostatic charge, a lower pKa, and the ability to remain molecularly dispersed and mostly dissociated at the pH of the vaginal lumen or below.

An acrylic based polymer such as, but not limited to, that shown in Formula II (poly (methyl vinyl ether/maleic anhydride) or MVE/MA) can be converted to its anhydride form which will allow for carboxylic acid and other substitutions to the polymer backbone. R′ in MVE/MA is a methyl group.

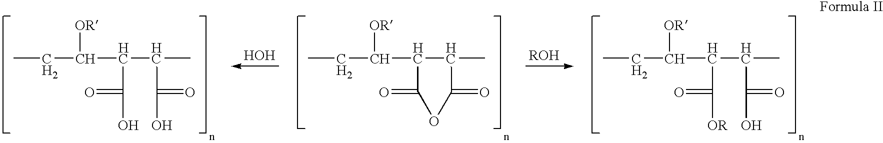

Formula II is based on any acrylic polymer or copolymer that has one or more dissociable carboxylic acid functions such that (R) of the polymer or copolymer backbone in Formula II can be substituted where the substituting agent is homogeneous or a heterogeneous mixture of —H, —CH₃, or —CH₂CH(OH)CH₃, or acetic acid, or any monocarboxylic acid, or it can be derived from trimellitic acid, or hydroypropyl trimellitic acid, or alternatively, R can be derived from any multi-carboxylic acid or a moiety containing sulfates, sulfonates, carboxylic acids or combinations of these groups as shown in (but not limited to) Table 1. The acid bearing moieties can be covalently attached to the polymer or oligomer backbone via several different mechanisms that one skilled in the art will appreciated including through an ester- linkage scheme using an alcohol intermediate (see FIG. 1).

The present invention includes safe and inexpensive compositions, formulations, and methods for treating or decreasing the spread of sexually transmitted diseases in a host comprising administering a therapeutically effective amount of a compound or compounds described in Formula I or Formula II, or their combinations.

In another aspect of this invention there is provided compositions and methods for treating infectious agents other than sexually transmitted diseases by topical application of a compound or compounds described in Formula I or Formula II.

In another aspect, there is provided a pharmaceutical formulation comprising a compound or compounds of the invention in Formula I or Formula II in combination with pharmaceutically acceptable carriers, emulsifiers, or excipients.

In still another aspect of this invention, there is provided a method for treating or decreasing the spread of sexually transmitted infection in a host by administering to the subject a combination comprising at least one compound according to Formula I or Formula II and at least one further anti-infective active agent or infection barrier agent such as a condom.

Another aspect of the invention is the use of a compound according to Formula I or Formula II for the preparation of a medicament for treating or preventing or decreasing the spread or transmission of viral infections, especially if the virus is one of the human immunodeficiency viruses or a member of the herpesvirus family, in the host.

In still another aspect of this invention, there is provided a method for treating or preventing or decreasing the spread or transmission of viral infections in a host, especially if the virus is one of the human immunodeficiency viruses, or a member of the herpesvirus family, by administering to the subject a combination comprising at least one compound according to Formula I or Formula II and at least one further therapeutic agent.

In still another aspect of this invention, there is provided a method for treating or preventing or decreasing the spread or transmission of viral infections in a host, especially if the virus is one of the human immunodeficiency viruses, or a member of the herpesvirus family, by administering to the subject a combination comprising at least one compound according to Formula I or Formula II and at least one further therapeutic agent that is derived from the polybiguanide (PBG) class of molecules.

In still another aspect of this invention, there is provided a method for treating or preventing or decreasing the spread or transmission of viral infections in a host, especially if the virus is one of the human immunodeficiency viruses, or a member of the herpesvirus family, by administering to the subject a combination comprising at least one compound according to Formula I or Formula II and at least one further therapeutic agent that is derived from the polybiguanide (PBG) class of molecules such as but not limited to polyethylene-hexamethylene biguanide (PEHMB).

The present invention also provides a safe and inexpensive method for treating or preventing the spread of bacterial or fungal infections in a host comprising administering topically a therapeutically effective amount of a compound or compounds described in Formula I or Formula II.

In still another aspect of this invention, there is provided a method for treating or preventing a bacterial or fungal infection in a host by administering to the subject a combination comprising at least one compound or compounds according to Formula I or Formula II and at least one further therapeutic agent. The compounds of this invention can be used in combination therapies with other classes of antiviral, antibacterial, or antifungal agent having similar or differing mechanisms of action including, but not limited to, anionic or cationic polymers or oligomers, surfactants, protease inhibitors, DNA or RNA polymerase inhibitors (including reverse transcriptase inhibitors), fusion inhibitors, cell wall biosynthesis inhibitors, integrase inhibitors, or virus or bacterial attachment inhibitors.

In still another embodiment of this invention, there is provided a method of use for a compound or compounds described in Formula I or Formula II, as an additive to cosmetic compositions.

In still another embodiment of this invention, there is provided a method for use of a compound or compounds described in Formula I or Formula II as an adjuvant that can be used in topical therapeutic and cosmetic formulations.

In still another embodiment of this invention, there is provided a method for use of a compound or compounds described in Formula I or Formula II as thickeners, alone or with other reagents, that can be used a vehicle for topical therapeutic and cosmetic formulations.

In still another embodiment of this invention, there is provided a method for use of a compound or compounds described in Formula I or Formula II as a disinfectant for use in eye drops, contact lens solutions, body washes, soaps, mouth washes, toothpastes, and other personal care and hygiene products.

In yet another embodiment, the present invention is directed to simultaneously tailoring the hydrophobicity of the resulting molecule, in addition to solubility and dissociation properties, by means of derivatization by both selecting the intermediate chemical structure and the level of its substitution in the polymer backbone. For the case of molecules having a cellulose-based backbone, the anhydride, acid chloride, or other reactive intermediate used to modify the polymers will include one or more aromatic (or heterocyclic) rings such that the resulting product would possess the right balance of solubility, hydrophobicity, and level of dissociable functional groups covering the pH range from 14 down to below 3.5, a condition necessary for desired biological activity in the acidic environment of the vaginal lumen with regard to retarding infectivity as elaborated in this invention.

Striking the balance between electrostatic and hydrophobic interaction in the resulting polymer is important to molecular binding of said polymer with gylcoproteins on viral and cellular surfaces. Interaction with viral surface proteins including gp120 and gp 41 of HIV-1 specifically requires both electrostatic and hydrophobic interactions to affect tight binding to the antiviral agent that would in turn prevent viral binding to cell surface receptors such CD4 or co-receptor like CCR5 and CXCR4. In order to achieve tight binding of antiviral agent to virus that in turn blocks infectivity of cells by said virus the antiviral agent polymer, copolymer or oligomer is preferably present in the molecularly dispersed state. Therefore, the presence of phenyl groups as in the case of trimellitic modifications is desirable for tailoring the hydrophobicity function of the molecule in order to enhance the desired biological activity. According to the present invention hydrophobicity can be imparted by the selecting an intermediate anhydride, or other equivalent modifying reagent, with a strong hydrophobic groups such as those bearing one or more aromatic rings including phenyl, naphthyl, and the like with known hydrophobic character. It is thus feasible to tailor the molecule with a smaller number of strong hydrophobic groups like naphthyl or a larger number of less hydrophobic groups like phenyl. One skilled in the art possesses the ability to strike the above balance between hydrophobicity, solubility and dissociation properties by manipulating the parameters of the modification and degree of substitution to arrive at the desired performance. The modifications according to the present invention are not limited to reactions with anhydrides but include any substitution of R at any of the —OH groups in the cellulosic backbone skeleton. It is thus highly desirable to have modified polymers bearing one or more hydrophobic groups such as phenyl and the like. Therefore the scope of the invention should not be limited by the discrete formulae or examples covered in the specification.

For acrylic based polymers, similar balance between hydrophobicity, solubility and dissociation is desirable to affect the biological function needed to suppress infectivity or STD transmission. For MVE/MA-like polymers, desired functional groups may be incorporated into the polymer either by selectively substituting the R group of vinyl co-monomer, or by reacting the resulting anhydride with the appropriate OH-bearing intermediates as shown in FIG. 1. It is thus feasible using a variety of strategies to incorporate moieties such as those shown in Table 1 into the acrylic polymer. For the purpose of the present invention, it is desirable to have molecularly dispersed polymer that remains dissociated in the pH range from 14 to below 3.5 and desirably possesses a level of hydrophobicity that would be optimal for blocking infectivity with STD causing agents.

It is yet another embodiment of the present invention to include both strong and weak acid groups in the polymer or copolymer, either cellulosic or acrylic such as those described in the specification. Weak acid groups include carboxylic groups having low pKs values as given in Table 1. Strong acid groups include sulfate, sulfonate, phosphate, or the like. Resulting molecules possessing the properties given in polymers such as HPMCT or acrylic equivalents and including strong acid groups such as sulfate and sulfonates will operate by more than one mechanism to prevent infectivity and transmission of STDs. The presence of sulfate groups in a polymeric molecule is known to strongly bind to the V3 loop of HIV-1 gp 120, and thus their incorporation to a molecule like HPMCT or similar molecules will expand the biological effectiveness of HPMCT by allowing the resultant molecule to function via more than one mechanism of action. The incorporation a sulfate or sulfonated group or moiety into a cellulose backbone is readily apparent to one skilled in the art and could be based upon the use of a compound such as, but not limited to the anhydride of 2-sulfobenzoic acid, as shown in Table 1.

In certain highly preferred embodiments of the invention the compositions of Formula I have the physical chemical capacity to remain molecularly dispersed in solution over a wide range of pH from 14 to preferably below 3.5.

The synthetic anionic polymeric polycarboxylates depicted in Formula II and FIG. 1, and employed herein are well known, being often employed in the form of their free acids or partially or fully neutralized water soluble alkali metal or ammonium salts (Nabi, N., and Gaffar, A. “Antibacterial, antiplaque oral composition.” U.S. Pat. No. 4,894,220 (1990)), or as the half ester as depicted in Formula II and FIG. 1. In another embodiment of this invention it is preferred that the polymeric polycarboxylates are 1:4 or 4:1 copolymers of maleic anhydride or acid with another polymerizable ethylenically unsaturated monomer, preferably methyl vinyl ether (yielding MVE/MA) having a molecular weight of about 500 to >2,000,000 most preferably about 10,000 to 250,000.

Other polymeric polycarboxylates that could be envisioned for use in Formula II include 1:1 copolymers of maleic anhydride with ethyl acrylate, hydroxyethyl methacrylate, N-vinyl-2-pyrrolidone, or ethylene, the later being available as Monsanto EMA No. 1103. In addition copolymers of acrylic acid with methyl or hydroxyethyl methacrylate, or methyl or ethyl acrylate, isobutyl vinyl ether or N-vinyl-2-pyrrolidone have also been described in the literature (Dichter; M., Mangaraj; D., King; W. J., James “Treatment of teeth.” U.S. Pat. No. 3,956,480 (1976)).

Still other polymeric polycarboyxlates that could be substituted for MVE/MA in Formula II include copolymers of maleic anhydride with styrene, isobutylene or ethyl vinyl ether, poly acrylic, polyitaconic and polymaleic acids and sulfoacrylic oligomers having molecular weights as low as 1,000 available as Uniroyal ND-2 (Gaffar; Maria Corazon S. “Composition to control mouth odor.” U.S. Pat. No. 4,138,477, (1979); Gaffar, A. and Gaffar, M. C. S., “Magnesium polycarboxylate complexes and anticalculuis agents.” U.S. Pat. No. 4,183,914 (1980)).

One skilled in the art will also realize that cross-linking the polymer of choice (such as MVE/MA) can lead to enhanced thickening or delivery aspects of the polymer by improving the viscoelastic properties of said polymer (Nabi; N., Prencipe; M., and Gaffar; A., “Antibacterial antiplaque oral composition.” U.S. Pat. No. 5,334,375, (1994)). Linearly viscoelastic compositions have excellent stability against phase separation or syneresis, viscosity change in storage, and settling of dissolved, dispersed or suspended particles under high and low temperature conditions, excellent texture and other cosmetic properties, ease of extrusion from a dispensing tube, pump or the like (easily shear thinned), good stand-up after extrusion. These types of compositions also have a high cohesive property, namely when a shear or strain is applied to a portion of the composition to cause it to flow, the surrounding portions will follow. As a result of this cohesiveness of the linear viscoelastic characteristic, the compositions will readily flow uniformly and homogeneously from a pump or tube when it is squeezed. The linear viscoelastic property also contributes to improved physical stability against phase separation upon storage.

For the purposes of this invention, if the above described polymers are to be cross-linked to be linearly viscoelastic then they should be lightly cross-linked so that they swell and form gels, strong three-dimensional networks in aqueous systems. Excessive cross-linking leads to hard, irreversible polymers and is to be avoided. The amount of cross-linking agent can vary from about 0.01 to about 30 weight percent of the total, cross-linked polymer, preferably about 2 to about 20 weight percent, more preferably about 3 to 15 weight percent.

### BRIEF DESCRIPTION OF THE DRAWINGS

FIG. 1. Synthesis of acrylic copolymers consisting of poly methyl vinyl ether and maleic anhydride (MVE/MA). The synthesis of MVE/MA involves the slow addition of molten maleic anhydride and methyl vinyl ether at 58° C. over a two hour period. The reaction is performed under pressure (e.g. 65 psi). The anhydride ring can be opened up to yield the corresponding half esters using an appropriate alcohol intermediate. Alternatively the dicarcoxylic acid can be achieved by the addition of H₂0. In addition the mono or mixed salt variants can be easily prepared. R′ in Formula II for MVE/MA is —CH₃

FIG. 2. Cytotoxicity evaluation of HPMCT in HeLa derived P4-R5 cells. Effect of varying doses of HPMCT (hydroxylpropyl methyl cellulose trimellitate), HPMCP (hydroxypropyl methyl cellulose phthalate), CAP (cellulose acetate phthalate, and CAT (cellulose acetate trimellitate) on uninfected P4-CCR5 cells are shown in FIG. 1. In this experiment test cells were exposed to HPMCT, HPMCP, CAP, or CAT, or the control compound Dextran Sulfate (DS) for two hours at 37° C. in 5% CO₂ atmosphere in tissue culture media. This is the standard amount of exposure that cells will receive in viral binding inhibition efficacy (VBI) assays shown in FIGS. 2 and 3. After drug exposure cells were washed and incubated in fresh drug-free medium for 48 hrs at 37° C. in 5% CO₂ atmosphere at which time the cells were assessed for viability using the MTT tetrazolium dye as described by Rando et al. (“Suppression of Human Immunodeficiency virus type 1 activity in vitro by oligonucleotides which form intramolecular tetrads”, J. Biol. Chem. 270:1754-1760 (1995)).

FIG. 3. Inhibitory effect of HPMCT, HPMCP, CAP, and CAT, on HIV-1IIIB, the CXCR4 tropic strain of HIV-1. Viral binding inhibition (VBI) assays were performed using P4-CCR5 cells treated with differing concentrations of HPMCT or the control compound DS for two hours in the presence of CXCR4 tropic HIV-1IIIB. The cells were then washed and incubated at 37° C. in drug and virus-free media for 48 hrs. At the end of the 48 hr culture the intracellular production of β-galactosidase (β-gal) was measured as described by Ojwang et al. (“T30177, an oligonucleotide stabilized by an intramolecular guanosine octet, is a potent inhibitor of laboratory strains and clinical isolates of human immunodeficiency virus type 1.” Antimicrobial Agents and Chemotherapy 39:2426-2435 (1995)). The decrease in β-gal production was measured relative to control infected but untreated cells.

FIG. 4. Effect of HPMCT on the CCR5 tropic HIV-1 strain BaL. VBI assays used P4-CCR5 cells treated with differing concentrations of HPMCT or the control compound DS for two hours in the presence of CCR5 tropic HIV-1BaL. The cells were then washed and incubated at 37° C. in drug and virus-free media for 48 hrs. At the end of the 48 hr culture the intracellular production of β-gal was measured as described by Ojwang et al. (“T30177, an oligonucleotide stabilized by an intramolecular guanosine octet, is a potent inhibitor of laboratory strains and clinical isolates of human immunodeficiency virus type 1.” Antimicrobial Agents and Chemotherapy 39:2426-2435 (1995)). The decrease in β-gal production was measured relative to control infected but untreated cells.

FIG. 5. Cell free virus inhibition (CFI) assay. In this CFI assay 8×10⁴ P4-CCR5 cells were plated in 12-well plates 24 hr prior to the assay. On the day of the assay, 5 μl of serially diluted compound was mixed with an equal volume of HIV-1IIIB (approximately 10⁴-10⁵ tissue culture infectious dose₅₀ (TCID₅₀) per μl) and incubated for 10 minutes at 37° C. After the incubation period, the mixture was diluted (100-fold in RPMI 1640 media including 10% FBS) and aliquots added to duplicate wells at 450 μl per well. After a 2-hr incubation period at 37° C., an additional 2 ml of new media was added to the cells. At 46 hr post-infection at 37° C., the cells were washed twice with phosphate buffered saline (PBS) and lysed using 125 μl lysis buffer (Galacto Star). HIV-1 infectivity (monitored by assaying for β-gal production) was measured by mixing 2-20 μl of centrifuged lysate with reaction buffer (Galacto Star), incubating the mixture for 1 hr at RT, and quantitating the subsequent luminescence.

FIG. 6. Cell associated virus inhibition (CAI) assay. In this assay, SupT1 cells (3×10⁶) were infected with HIV-1IIIB in RPMI media (30 μl) and incubated for 48 hr. Infected SupT1 cells were pelleted and then resuspended (8×10⁵ cells/ml) in tissue culture media. Differing concentrations of HPMCT (5 μl) were added to infected SupT1 cells (95 μl) and incubated for 10 min at 37° C. After incubation, the mixture was diluted in RPMI media (1:10) and 300 μl was added to appropriate wells in triplicate. In the wells, target P4-CCR5 cells were present. Production of infectious virus will result in b-gal induction in the P4-CCR5 targets. Plates are incubated (2 hr at 37° C.), washed (2×) with PBS and then media (2 ml) is added and before further incubation (22-46 hr). Cells are then aspirated and washed (2×) and then incubated (10 min at room temperature) with lysis buffer (125 μl). Cell lysates are assayed for β-gal production utilizing the Galacto-Star™ kit (Tropix, Bedford, Mass.).

FIG. 7. Combination studies using HPMCT and PEHMB. HPMCT was added over a range of concentrations combined with 0.01% polyethylene hexamethylene biguanide or PEHMB (Catalone, B. J., et al. “Mouse model of cervicovaginal toxicity and inflammation for the preclinical evaluation of topical vaginal microbicides”, Antimicrob. Agents and Chemother. 2004 in press) to P4-CCR5 cells in a VBI assay (FIG. 6A). Reverse experiments were also performed in which 0.0002% HPMCT was used in combination with various concentrations of PEHMB (FIG. 6B). In these assays a 1.0% wt/vol stock solutions of HPMCT dissolved in 20 mM sodium citrate buffer pH 5.0, and a 5% PEHMB wt/vol solution made up in saline were used.

FIG. 8. HSV-2 plaque reduction assay. HSV-2 (strain 333) virus stocks were prepared at a low multiplicity of infection with African Green monkey kidney (CV-1) cells, and subsequently, cell-free supernatants were prepared from frozen and thawed preparations of lytic infected cultures. CV-1 cells were seeded onto 96-well culture plates (4×104 cell/well) in 0.1 ml of minimal essential medium (MEM) supplemented with Earls salts and 10% heat inactivated fetal bovine serum and pennstrep (100 U/ml penicillin G, 100 μg/ml streptomycin sulfate) and incubated at 37° C. in 5% CO2 atmosphere overnight. The medium was then removed and 50 μl of medium containing 30-50 plaque forming units (PFU) of virus dilute d in test medium and various concentrations of HPMCT were added to the wells. Test medium consisted of MEM supplemented with 2% FBS and pennstrep. The virus was allowed to adsorb to the cells in the presence of HPMCT for 1 hr. The test medium was then removed and the cells were rinsed three times with fresh medium. A final 100 μl aliquot of test medium was added to the cells which were then further cultured at 37° C. Cytopathic effect was scored 24 to 48 hrs post infection when control wells showed maximum effect of virus infection. Each datum in FIG. 6 represents an average for at least two plates.

### DETAILED DESCRIPTION OF THE INVENTION

The present invention involves the use of cellulose, acrylic (or other) polymer or copolymer or oligomer backbone-derived agent, such that the oligomer or polymer or copolymer is able to remain molecularly dispersed and mostly dissociated in an aqueous environment over a pH range of 14 to preferably below 3.5. These molecules will have multiple applications including but not limited to the use to treat or reduce the spread of infectious organisms such as sexually transmitted diseases (STDs).

The polymers or oligomers of this invention are usually, but not always, substituted with moieties containing one or more carboxylic acid, sulfate or sulfonate group or mixtures of these groups therein. The degree of substitution (homogeneous or heterogeneous) per repeat unit of the indicated polymer, copolymer, or oligomer is such that the resulting molecule is able to remain soluble (molecularly dispersed) and mostly dissociated at the pH range encountered in the vaginal lumen. For example, HPMCT has been reported to have been synthesized with various levels of trimellityl, hydroxypropoxyl, and methoxyl substitution ranging from 0.28 to 0.66 units trimellityl per unit of glucose. However, only when the right combination of the three substituents was achieved did the resulting molecule dissolve in an aqueous solution at pH 4.0 or below (Kokubo, H., et al., “Development of Cellulose derivatives as novel enteric coating agents soluble at pH 3.5-4.5 and higher.” Chem. Pharm. Bul1 45:1350-1353 (1997)). The size of the oligomers or polymers or copolymers can vary from as low as 500 daltons to >2 MM daltons, and the pKa of the resultant molecule must be low enough to allow for one or more free acid groups to remain dissociated at pH values in solution of 3.5 or less. The dissociated acidic groups of the invention are important for both the solubility and biologic activity of the molecule. For example the pH in the vaginal lumen is in the range of 3.4 to 6.0 (S. Voeller, D. J. Anderson, “Heterosexual Transmission of HIV.” JAMA 267, 1917-1918 (2000)), and may undergo a transient increase in pH upon the addition of semen which has a pH of about 8.0. In addition, the mechanism by which CAP inactivates HIV-1 is through a direct binding to HIV-1 gp120 (Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120.” BMC Infectious Diseases 1:17 (2001); Neurath, A. R., Strick, N., Jiang, S., Li, Y. Y., and Debnath, A. K. “Anti-HIV-1 activity of cellulose acetate phthalate: Synergy with soluble CD4 and induction of “dead-end” gp41 six-helix bundles.” BMC Infectious Diseases 2:6 (2002)). It is believed that CAP interacts with the V3 loop of gp120 using both electrostatic (hydrogen bonding with arginines at amino acid positions 311 and 315) and hyrdrophobic forces contacting phenylalanine at amino acid position 317 (Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120.” BMC Infectious Diseases 1:17 (2001)). CAP is also postulated to interact directly with HIV-1 gp41, again using both electrostatic (at gp41 amino acid 579, which is an arginine) and hydrophobic (gp41 position 571 which is tryptophan) forces (Neurath, A. R., Strick, N., Jiang, S., Li, Y. Y., and Debnath, A. K. “Anti-HIV-1 activity of cellulose acetate phthalate: Synergy with soluble CD4 and induction of “dead-end” gp41 six-helix bundles.” BMC Infectious Diseases 2:6 (2002)). Therefore, for the development of an effective microbicide to prevent or decrease the spread of a STD it is desirable to have an agent that remains in its molecularly dispersed state in solution and maintains its biological activity in the entire pH range that would be encountered under these physiologic conditions (i.e. approximately 3.0 to >8.0). In addition, the molecule must remain in a dissociated state in order to be capable of interacting via electrostatic forces, especially within the vaginal pH range. The remaining free carboxylic acid group in CAP has a pKa of about 5.3 and thus it will not be dissociated in the pH of the vaginal environment.

Polymers, copolymers or oligomers having carboxyl groups that are not dissociated have very low solubility in water; as the pH is raised equilibrium shifts to the formation of the ionized form with increasing water solubility. Thus, the pH at which cellulosic polymers become soluble can be controlled by adjusting both the kind of carboxylic acid moiety linked to the polymer or oligomer backbone, and the degree of substitution. The present invention involves the use of carboxylic acid substituted oligomers or polymers which retain their solubility at pH <3.5 (that is they remain molecularly dispersed and mostly dissociated in solution) to retard or prevent the transmission of infectious disease and to prevent, retard, or treat sexually transmitted diseases. In addition these oligomers or polymers can be used in combination therapies to treat STDs and other infectious organisms, as additives or as an adjuvant to other therapeutic formulations, as a plasticizer, as part of a cosmetic formulation, as a disinfectant for general household or industrial use, as an active agent to reduce bacterial, viral or fungal contamination in ophthalmic applications such as eye drops or contact lens solutions, and in toothpaste or mouthwash formulations. Combination therapy is, as its name implies, the use of two or more agents simultaneously for the purpose of obtaining a better therapeutic outcome than could be obtained using only one agent (monotherapy). A better therapeutic outcome would include a reduced risk of spread of a sexually transmitted disease upon use of the combination therapy. For use in the prevention of spread of a STD the combination might include one or more topical agent administered simultaneously or in some defined pattern. In addition, the combination therapy might include the administration of one or more topical therapeutic agents along with one or more agents that have a differing route of administration (such as via an injection or an oral route of administration). The compounds of this invention can be used in combination therapies with other classes of antiviral, antibacterial, or antifungal agent having similar or differing mechanisms of action including, but not limited to, anionic or cationic polymers or oligomers, surfactants, protease inhibitors, DNA or RNA polymerase inhibitors (including reverse transcriptase inhibitors), fusion inhibitors, cell wall biosynthesis inhibitors, integrase inhibitors, or virus or bacterial attachment inhibitors.

In 1997 Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)) demonstrated that by careful selection of the carboxylic acid containing moiety used to link with a cellulose-based polymer backbone, the overall pKa of the polymer could be tailored to fit specific needs. The thrust of this work by Kokubo was to obtain superior enteric coating agents and not new anti-infective agents. In addition in 2000 Neurath reported that CAP and HMPCP are effective agents against sexually transmitted diseases (Neurath A. R. et al. “Methods and compositions for decreasing the frequency of HIV, herpsevirus and sexually transmitted bacterial infections.” U.S. Pat. No. 6,165,493, (2000)). In that study Neurath's group appreciated the fact that carboxylic acid groups of CAP and HPMCP were not entirely dissociated at the vaginal pH, and that the two compounds were insoluble under such pH conditions. Further, Neurath's group actually propose to use micron size particulate formulations of their identified compounds to help get around the solubility issue (Neurath A. R. et al. “Methods and compositions for decreasing the frequency of HIV, herpsevirus and sexually transmitted bacterial infections.” U.S. Pat. No. 6,165,493, (2000); Manson, K. H. et al. “Effect of a Cellulose Acetate Phthalate Topical Cream on Vaginal Transmission of Simian Immunodeficency Virus in Rhesus Monkeys.” Antimicrobial Agents and Chemotherapy 44:3199-3202 (2000)). It is not clearly understood how micronized particles work as microbicides at the vaginal pH, since most of the testing was performed in vitro at the neutral pH of approximately 6.5 to 7.5 (Neurath A. R. et al. “Methods and compositions for decreasing the frequency of HIV, herpes virus and sexually transmitted bacterial infections.” U.S. Pat. No. 6,165,493, (2000)). It is likely that the particles act via an adsorption mechanism by binding virus that comes into contact with it. Therefore, the use of chemical moieties to enhance the low pH solubility and significant dissociation of the ionizable functional groups of cellulosic-based or other polymers and then using those polymers as anti-infective agents was not taught by prior inventors.

In one embodiment of the present invention cellulose based polymers such as HPMCT, HPMCP, CAT, and CAP are further derivitized by the addition of a sulfate or sulfonate or other strong acid group to a free hydroxyl on the polymer for the purpose of increasing the solubility (molecular dispersed in solution) and dissociation of the functional group over a wide range of pH from 14 to below 3.5. These modifications will increase the overall biological effectiveness of the agent under physiologic conditions encountered in the vaginal lumen.

In one embodiment of the present invention the infectious agent is selected from the group consisting of human immunodeficiency virus types 1 and 2.

In another embodiment, the retrovirus infection is human immunodeficiency virus type 1 (HIV-1).

In one embodiment of the invention the viral infection is selected from the group consisting of herpes virus infections.

In another embodiment, the herpes virus is selected from the group consisting of herpes simplex virus type 1 (HSV1) and herpes simplex virus type 2 (HSV2).

In another embodiment, the herpes virus is herpes simplex virus type 2 (HSV2).

In one embodiment of the present invention the infectious agent is bacterial in origin.

In another embodiment, the bacterial species is Trichomonas vaginalis, Neisseris gonorrhoeae Haemopholus ducreyi, or Chlamydia trachomatis.

In another embodiment, the sexually transmitted disease would consist of one of the following microorganisms identified as causative agents in bacterial vaginosis, Gardnerella vaginalis, Mycoplasma hominis, Mycoplasma capricolum, Mobiluncus curtisii and Prevotella corporis.

In another embodiment, the infectious disease would consist of a microorganisms identified as causing infection in ophthalmic, cutaneous, or nasopharyngeal or oral anatomic sites.

In one embodiment, the compounds and methods of the present invention comprise those wherein the following embodiments are present, either independently or in combination:

In one aspect of the present invention, R in Formula I or Formula II is an aliphatic or aromatic moiety containing more than one carboxylic acid groups such that once covalently attached to the polymer, copolymer, or oligomer backbone the resultant compound can remain molecularly dispersed and mostly dissociated in solution at a range of pH from 14 to below 3.5.

In other aspect the oligomer or polymer in Formula I is hydroxylpropyl methyl cellulose (HPMC)—based.

In other aspect the oligomer or polymer in Formula I is cellulose acetate based.

In another aspect R in Formula I is derived from reaction with trimellitic anhydride and the resultant molecule is hydroxypropyl methylcellulose trimellitate, abbreviated HPMCT.

In another aspect R in Formula I is derived from reaction with a mixture of maleic anhydride and acetic acid and the resultant molecule is hydroxypropyl methylcellulose acetate maleate, abbreviated HPMC-AM.

In another aspect R in Formula I is derived from reaction with a mixture of 2-sulfobenzoic acid cyclic anhydride and acetic acid and the resultant molecule is hydroxypropyl methylcellulose acetate sulfobenzoate.

In another aspect R in Formula I is derived from reaction with a mixture of trimellitic anhydride and acetic acid and the resultant molecule is cellulose acetate trimellitate, abbreviated CAT.

In another aspect R in Formula I is derived from reaction with a mixture of 2-sulfobenzoic acid cyclic anhydride and acetic acid and the resultant molecule is cellulose acetate sulfobenzoate.

In another aspect R in Formula I is derived from reaction with a mixture of 2-sulfobenzoic acid cyclic anhydride and acetic acid and, a second anhydride such as an anhydride derived from phthalic or trimellitic acid and the resultant compound can remain molecularly dispersed and mostly dissociated in solution at a range of pH from 14 to below 3.5.

In other aspect the oligomer or polymer in Formula II is acrylic-based.

In other aspect the oligomer or polymer in Formula II is a copolymer of methylvinyl ether and maleic anhydride or other acrylic analogue.

In another aspect R in Formula I is —H, OH, or —CH₃, or —CH₂CH(OH)CH₃ or similar moiety.

In another aspect R in Formula I or Formula II is a single carboxylic acid containing moiety like but not limited to acetic acid.

In another aspect R in Formula I or Formula II is selected from, but not limited to, the multi-carboxylic acid containing moieties shown in Table 1.

In a further embodiment, the present invention relates to a method for the treatment or prevention of virus, bacterial, or fungal infections using cellulose or acrylic-based compound oligomers or polymers and administering a therapeutically effective amount of said compound having the general structure found in Formulas I or II, or a pharmaceutically acceptable salt or formulation thereof, alone or in combination with a second active anti-infective agent:

Wherein R in Formula I or Formula II can be a mixture of —H, or —CH₃, or —CH2CH(OH)CH₃, or derived from acetic acid, or any monocarboxylic acid, and (in defined proportions) moieties derived from trimellitic acid, or hydroypropyl trimellitic acid, or any di- or tri-, or multi-carboxylic, sulfonic, or sulfate derived acid as shown in (but not limited to) Table 1 such that upon covalent addition to the cellulose or acrylic polymer backbone the resultant molecule is able to remain molecularly disperse and mostly dissociated in aqueous solutions in which the pH is ranges between 14 to below 3.5.

In yet another embodiment, the present invention is directed to simultaneously tailoring the hydrophobicity of the resulting molecule, in addition to solubility and dissociation properties, by both selecting the intermediate chemical structure and the level of its substitution in the polymer backbone. For the case of molecules having a cellulosic-based backbone, the anhydride, acid chloride, or other reactive intermediate used to derivatize the polymers will include one or more aromatic (or heterocyclic) rings such that the resulting product would possess the right balance of solubility, hydrophobicity, and level of dissociable functional groups covering the pH range from 14 down to below 3.5, a condition necessary for desired biological activity in the acidic environment of the vaginal lumen with regard to retarding infectivity as elaborated in this invention. We have found a balance between solubility, dissociation and hydrophobicity for the case of HPMCT to be in the range of 0.25 to 0.7 trimellityl substituents per glucose unit. That is to say an HPMC chain 100 glucose units in length will have optimally 25 to 70 trimellityl substituents. Equivalent molecules can be tailored to exhibit the balance of properties that we were able to obtain in HPMCT.

Striking the balance between the ability to reaming in the dissociated state over a wide range of pH, electrostatic, and hydrophobic interactions in the resulting polymer (copolymer or oligomer) is important to molecular binding of said molecule with gylcoproteins on viral and cellular surfaces. Interaction with viral or cellular surface proteins may require both electrostatic and hydrophobic forces to affect tight binding as has been reported for CAP (Neurath, A. R., Strick, N., Jiang, S., Li, Y. Y., and Debnath, A. K. “Anti-HIV-1 activity of cellulose acetate phthalate: Synergy with soluble CD4 and induction of “dead-end” gp41 six-helix bundles.” BMC Infectious Diseases 2:6 (2002); Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120.” BMC Infectious Diseases 1:17 (2001)). Therefore, the presence of phenyl groups as in the case of trimellitic modifications is desirable for tailoring the hydrophobicity function of the molecule in order to enhance the desired biological activity. According to the present invention hydrophobicity can be imparted by the selecting an intermediate anhydride, or other equivalent modifying reagent, with a strong hydrophobic groups such as those bearing one or more aromatic rings including phenyl, naphthyl, and the like with know hydrophobic character. It is thus feasible to tailor the molecule with a smaller number of strong hydrophobic groups like naphthyl or a larger number of less hydrophobic groups like phenyl. One skilled in the art possesses the ability to strike the above balance between hydrophobility, solubility and dissociation properties by manipulating the parameters of the modification and degree of substitution to arrive at the desired performance. The modifications according to the present invention are not limited to reactions with anhydrides but include any substitution of R at any of the —OH groups in the cellulosic backbone skeleton. It is thus highly desirable to have modified polymers bearing one or more hydrophobic groups such as phenyl and the like. We have found that such balance could be made in the case of HPMCT at a range of trimellityl substitution of 0.25 to 0.7 per glucose unit. This balance and subsequent biological activity could be duplicated with other modifiers by changing conditions and level of substitution. Therefore the scope of the invention should not be limited by the discrete formulae or examples covered in the specification.

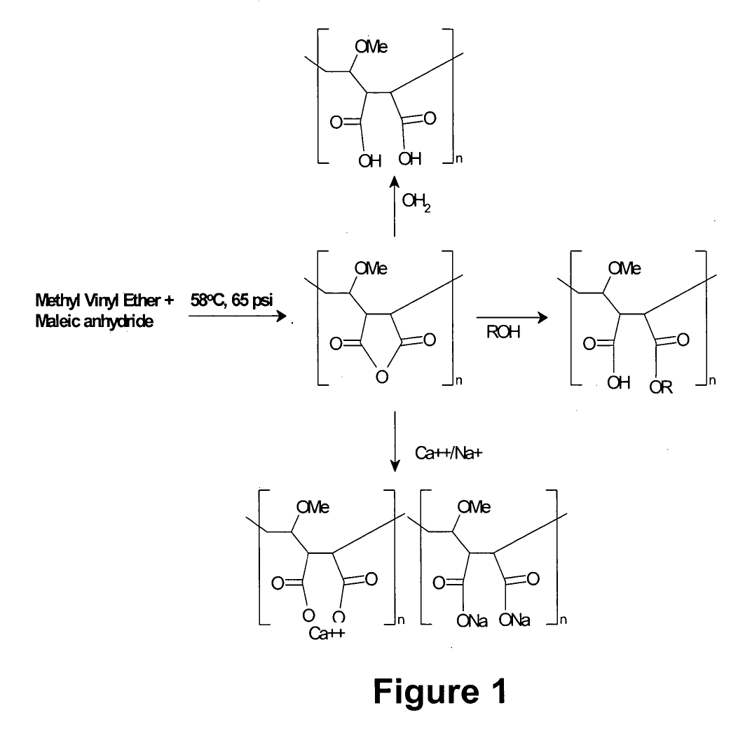

For acrylic based polymers, similar balance between hydrophobicity, solubility and dissociation is desirable to affect the biological function needed to suppress infectivity or STD transmission. For example, in MVE/MA-like polymers, desired functional groups may be incorporated into the polymer either by selectively substituting the R group of the vinyl co-monomer used, or by mixing under the proper conditions the resulting anhydride with the appropriate R—OH-bearing intermediates as shown in FIG. 1. It is thus feasible using a variety of strategies to incorporate moieties such as those shown in Table 1 into the acrylic-based polymer. For the purpose of the present invention, it is desirable to have molecularly dispersed polymer that remains dissociated in the pH range from 14 to below 3.5 and desirably possesses a level of hydrophobicity that would be optimal for blocking infectivity with STD causing agents. Further, introduction of sulfate or sulfonate groups, or other groups with low pKa values should bring favorable solubility and dissociation parameters to very low pH levels (e.g. ≦1.0). Therefore, one skilled in the art could manipulate the reaction to achieve the latter result.

It is yet another embodiment of the present invention to include both strong and weak acid groups in the polymer or copolymer, either cellulosic- or acrylic-based such as those described in the specification. Weak acid groups include carboxylic groups having low pKas values as given in Table 1. Strong acid groups include sulfate, sulfonate, phosphate, or others with low pKas in the range of 1.0 or below. Resulting molecules possessing the properties given in polymers such as HPMCT or acrylic equivalents and including strong acid groups such as sulfate and sulfonates will operate by more than one mechanism to prevent infectivity and transmission of STDs. The presence of sulfate groups in a polymeric molecule is know to strongly bind to the V3 loop of HIV-1 gp 120 (Esté, J. A., Schols, D., De Vreese, K., Cherepanov, P., Witvrouw, M., Pannecouque, C., Debyser, Z., Desmyter, J., Rando, R. F., and De Clercq, E., “Human immunodeficiency virus glycoprotein gp120 as the primary target for the antiviral action of AR177 (Zintevir).” Mol. Pharm. 53:340-345 (1998)), and thus the addition of sulfate or sulfonate groups to a molecule like HPMCT, or similar molecules, will expand the spectrum of activity by conferring to the new molecule the ability to act via to multiple distinct mechanisms. The incorporation a sulfate or sulfonated moiety into a cellulose backbone is readily apparent to one skilled in the art and could be based upon the use of a compound such as, but not limited to, the anhydride of 2-sulfobenzoic acid, as shown in Table 1. The incorporation a sulfate or sulfonated moiety into a cellulose backbone along with carboxylic acid groups is readily apparent to one skilled in the art and could be based upon the use of a compound such as, but not limited to the anhydride of 4-sulfo-1,8-naphthalic acid, as shown in Table 1. Furthermore, the position of the sulfate or sulfonate groups on the ring structures can be varied to adjust performance of the resulting polymer.

In a further embodiment, the present invention relates to a method for the treatment or prevention of virus infections using a cellulose or acrylic-based oligomer or polymer (like but not limited to HPMCT, HPMCAM, or MVE/MA or other acrylic-based material) compounds soluble and mostly dissociated over a wide range of pH (i.e. 14 to below 3.5), administering a therapeutically effective amount of said compound or a pharmaceutically acceptable salt thereof, wherein the virus is selected is a retrovirus.

In a further embodiment, the present invention relates to a method for the treatment or prevention of virus infections using a cellulose or acrylic-based oligomer or polymer (like but not limited to HPMCT, HPMCAM, or MVE/MA or other acrylic-based material) compounds soluble and mostly dissociated over a wide range of pH (i.e. 14 to below 3.5), administering a therapeutically effective amount of said compound or a pharmaceutically acceptable salt thereof, wherein the virus is the human immunodeficiency virus type 1 (HIV-1).

In a further embodiment, the present invention relates to a method for the treatment or prevention of virus infections using a cellulose or acrylic-based oligomer or polymer (1 like but not limited to HPMCT, HPMCAM, or MVE/MA or other acrylic-based material) compounds soluble and mostly dissociated over a wide range of pH (i.e. 14 to below 3.5), administering a therapeutically effective amount of said compound or a pharmaceutically acceptable salt thereof, wherein the virus is selected is a member of the herpes virus family.

In a further embodiment, the present invention relates to a method for the treatment or prevention of virus infections using a cellulose or acrylic-based oligomer or polymer (like but not limited to HPMCT, HPMCAM, or MVE/MA or other acrylic-based material) compounds soluble and mostly dissociated over a wide range of pH (i.e. 14 to below 3.5), administering a therapeutically effective amount of said compound or a pharmaceutically acceptable salt thereof, wherein the virus is herpes simplex virus type 2 (HSV2).

In a further embodiment, the present invention relates to a method for the treatment or prevention of virus infections using a cellulose or acrylic-based oligomer or polymer (like but not limited to HPMCT, HPMCAM, or MVE/MA or other acrylic-based material) compounds soluble and mostly dissociated over a wide range of pH (i.e. 14 to below 3.5), administering a therapeutically effective amount of said compound or a pharmaceutically acceptable salt thereof, wherein the infectious agent is bacterial or fungal in origin.

In a further embodiment, the present invention relates to a method for the treatment or prevention of virus infections using a cellulose or acrylic-based oligomer or polymer (like but not limited to HPMCT, HPMCAM, or MVE/MA or other acrylic-based material) compounds soluble and mostly dissociated over a wide range of pH (i.e. 14 to below 3.5), administering a therapeutically effective amount of said compound or a pharmaceutically acceptable salt thereof, wherein the infectious agent is one or more of the following: Trichomonas vaginalis, Neisseris gonorrhoeae Haemopholus ducreyi, or Chlamydia trachomatis, Gardnerella vaginalis, Mycoplasma hominis, Mycoplasma capricolum, Mobiluncus curtisi, Candida albicans, and/or Prevotella corporis.

There is also provided pharmaceutically acceptable salts of the compounds of Formula I of the present invention. By the term pharmaceutically acceptable salts of the compounds of Formula I are meant those derived from pharmaceutically acceptable inorganic and organic acids such as alkali metals sodium and potassium or equivalent organic cation.

The term “host” represents any mammals including humans.

In one embodiment, the host is human.

The compounds of the present invention can be prepared by methods well known in the art. The synthesis of some of the cellulose-based compounds have been previously described by Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)) and by Kokubo and Nishiyama (“Aqueous coating composition and process for preparing solid pharmaceutical preparations.” U.S. Pat. No. 6,258,799 (2001); and Japanese Patent JP-A 8-301790).

Acrylic copolymers such as MVE/MA and other acrylic based materials are easily prepared from starting materials such as methyl vinyl ether and maleic anhydride. It should be obvious to one skilled in the art of organic or polymer chemistry that there are multiple different routes for preparing compounds as described in Formulas I and II, including but not limited to the creation of an ester or ether linkage using anhydride and alcohol containing intermediates.

According to one embodiment, it will be appreciated that the amount of a compound of Formula I or II of the present invention required for use in therapeutic treatment will vary not only with the particular compound selected but also with the route of administration, the nature of the condition for which treatment is required and the age and condition of the patient and will be ultimately at the discretion of the attendant physician or veterinarian. In general however a suitable dose will range from about 0.01 to about 750 mg/kg of body weight per day, preferably in the range of 0.5 to 60 mg/kg/day, most preferably in the range of 1 to 20 mg/kg/day for systemic administration, or for topical applications a suitable dose will range from about 0.001 to 25% wt/vol, preferably in the range of 0.001 to 5% wt/vol of formulated material. If the material is to be micro-dispersed (micronized) instead of molecularly dispersed in solution, and applied thus, then the effective amount of the dose could range from 0.01 to 25 weight percent of micronized cellulosic- or acrylic-based polymer or oligomer derivative.

The desired dose according to one embodiment is conveniently presented in a single dose or as a divided dose administered at appropriate intervals, for example as two, three, four or more doses per day.

While it is possible that for use in therapy a compound of Formula I or II of the present invention may be administered as a single agent molecularly dispersed in an aqueous solution, it is preferable according to one embodiment of the invention, to present the active ingredient as a pharmaceutical formulation. The embodiment of the invention thus further provides a pharmaceutical formulation comprising a compound of Formula I or II or a pharmaceutically acceptable salt thereof together with one or more pharmaceutically acceptable carriers thereof and, optionally, other therapeutic and/or prophylactic ingredients. The carrier(s) must be “acceptable” in the sense of being compatible with the other ingredients of the formulation and not deleterious to the recipient thereof.

According to one embodiment of the present invention, pharmaceutical formulations include but are not limited to those suitable for oral, rectal, nasal, topical, (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods according to this embodiment include the steps of bringing into association the active compound with liquid carriers or finely divided solid carriers or both and then, if necessary, shaping the product into the desired formulation.

According to another embodiment, pharmaceutical formulations suitable for oral administration are conveniently presented as discrete units such as capsules, cachets or tablets each containing a predetermined amount of the active ingredient, as a powder or granules. In another embodiment, the formulation is presented as a solution, a suspension or as an emulsion. In still another embodiment, the active ingredient is presented as a bolus, electuary or paste. Tablets and capsules for oral administration may contain conventional excipients such as binding agents, fillers, lubricants, disintegrants, or wetting agents. The tablets may be coated according to methods well know in the art. Oral liquid preparations may be in the form of, for example aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds in Formula I or II according to an embodiment of the present invention are formulated for parenteral administration (e.g. by bolus injection or continuous infusion) and may be presented in unit dose form in ampoules, pre-filled syringes, small volume infusion or in multi-dose containers with an added preservative. The compositions may take such forms as suspensions, solutions, emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

For topical administration to the epidermis (mucosal or cutaneous surfaces), the compounds of Formula I or II, according to one embodiment of the present invention, are formulated as ointments, creams or lotions, or as a transdermal patch. Such transdermal patches may contain penetration enhancers such as linalool, carvacrol, thymol, citral, menthol, and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Pharmaceutical formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

In another embodiment of the present invention a pharmaceutical formulation suitable for rectal administration consists of the active ingredient and a carrier wherein the carrier is a solid. In another embodiment, they are presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

According to one embodiment, the formulations suitable for vaginal administration are presented as pessaries, tampons, creams, gels, pastes, foams, or sprays containing in addition to the active ingredient such carriers as are known in the art to be appropriate.

According to another embodiment, the formulations suitable for vaginal administration can be delivered in a liquid or solid dosage form and can be incorporated into barrier devices such as condoms, diaphragms, or cervical caps, to help prevent the transmission of STDs

For intra-nasal administration the compounds, in one embodiment of the invention, are used as a liquid spray or dispersible powder or in the form of drops. Drops may be formulated with an aqueous or non-aqueous base also comprising one or more dispersing agent, solubilising agent, or suspending agent. Liquid sprays are conveniently delivered from pressurized packs.

For administration by inhalation the compounds in Formula I or II, according to one embodiment of the invention are conveniently delivered from an insufflator, nebulizer or pressurized pack or other convenient means of delivering an aerosol spray.

In another embodiment, pressurized packs comprise a suitable propellant such as dichlorodifluoromethane, trichlorofluoromethane, dichlorotetrafluoroethane, carbon dioxide or other suitable gas.

In another embodiment, the dosage unit in the pressurized aerosol is determined by providing a valve to deliver a metered amount.

Alternatively, in another embodiment, for administration by inhalation or insufflation, the compounds of Formula I or II according to the present invention are in the form of a dry powder composition, for example a powder mix of the compound and a suitable powder base such as lactose or starch. In another embodiment, the powder composition is presented in unit dosage form in, for example, capsules or cartridges or e.g. gelatin or blister packs from which the powder may be administered with the aid of an inhalator or insufflator.

In one embodiment, the above-described formulations are adapted to give sustained release of the active ingredient.

The compounds of the invention may also be used in combination with other antiviral agents that have already been approved by the appropriate governmental regulatory agencies for sale or are currently in experimental clinical trial protocols.

In one embodiment, the compounds of the invention may be employed together with at least one other antiviral agent chosen from a list that includes but is not limited to antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors or virus or bacterial attachment inhibitors.

In one embodiment, the compounds of the invention may be employed together with at least one other antiviral agent chosen from amongst agents approved for use in humans by government regulatory agencies.

In one embodiment, the compounds of the invention may be employed together with at least one other antiviral agent chosen from amongst approved HIV-1 RT inhibitors (such as but not limited to, Tenofovir, epivir, zidovudine, or stavudine, etc.), HIV-1 protease inhibitors (such as but not limited to saquinavir, ritonavir, nelfinavir, indinavir, amprenavir, lopinavir, atazanavir, tipranavir, or fosamprenavir), HIV-1 fusion inhibitors (such as but not limited to Fuzeon (T20), or PRO-542, or SCH—C), and a new or emerging classes of agents such as the positively charged class of polymers and oligomers know as polybiguanides (PBGs). In addition compounds of this invention can also be used in combination with other polyanionic compounds especially those bearing a sulfate or sulfonate group.

In one embodiment, the compounds of the invention may be employed together with at least one other antiviral agent chosen from amongst herpes virus DNA polymerase inhibitors (such as acyclovir, ganciclovir, cidofovir, etc.), herpes virus protease inhibitors, herpes virus fusion inhibitors, herpes virus binding inhibitors, and/or ribonucleotide reductase inhibitors.

In one embodiment, the compounds of the invention may be employed together with at least on other antiviral agent chosen from Interferon-α and Ribavirin, or together with a combination of Ribavirin and Interferon-α.

In a further embodiment, the compounds of the invention may be employed together with at least one other anti-infective agent know to be effective against but not limited to any of the following bacterial or fungal organisms: Trichomonas vaginalis, Neisseris gonorrhoeae Haemopholus ducreyi, or Chlamydia trachomatis, Gardnerella vaginalis, Mycoplasma hominis, Mycoplasma capricolum, Mobiluncus curtisii and Prevotella corporis, Calymmatobacterium granulomatis, Treponema pallidum, and Candida albicans.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The individual compounds of such combinations may be administered either sequentially or simultaneously in separate or combined pharmaceutical formulations.

When the compound of Formula I or II, or a pharmaceutically acceptable salt or formulation thereof is used in combination with a second therapeutic agent active against the same or different virus, the same or different strain of bacteria, or the same or different type of fungal infection, the dose of each compound may either be the same as or differ from that when the compound is used alone. Appropriate doses will be readily appreciated by those skilled in the art, or by the attending physician.

Acrylic and cellulose based polymers or copolymers can also be chemically cross-linked to varying degrees to improve their linear viscoelastic properties.

The following examples are provided to illustrate various embodiments of the present invention and shall not be considered as limiting in scope.

### EXAMPLES

### Example 1

### Synthesis of Acrylic-Based Polymers, Copolymers or Oligomers

Acrylic based polymers and copolymers can be obtained using a variety of techniques that would be apparent to one skilled in the art. For example, a synthetic scheme that one could employ to synthesize MVE/MA involves the addition of 404.4 parts cyclohexane, and 269.6 parts ethyl acetate into a I liter pressure reactor. Next 0.3 parts of t-butylperoxypivilate are added at 58° C. in three installments of 0.1 part each at times 0, 60 and 120 minutes from the first addition. Seventy-five parts of molten maleic anhydride and 49.0 parts of methyl vinyl ether are mixed together and gradually added to the reaction vessel at 58° C. and 65 psi over a 2 hour period of time. The reaction mixture is then held at 58° C. for two hours after the last addition of initiator. The presence of maleic anhydride is followed by testing with triphenyl phosphene. The product precipitates out of solution. After the reaction is complete the product is cooled to room temperature, filtered and dried in a vacuum oven. If cross-linked copolymer is desired then add (for example) 6 parts of 1,7 octadiene to the reaction vessel before the addition of the t-butylperoxypivilate.

### Example 2

### Derivitization of Acrylic-Based Polymers, Copolymers or Oligomers to Achieve Enhanced Solubility at Low pH

One skilled in the art could imagine several different mechanisms for creating diversity within the acrylic polymer or copolymer motif that will allow for variation in charge density or hydrophobicity. One mechanism would be to interchange maleic anhydride in Example 1 above with any anhydride derivative of moieties containing one or more carboxylic acid group as shown in, but not limited to, Table 1. Alternatively a mixture of two or more anhydride containing moieties, derived from examples shown in Table 1, could be used to generate a polymer with alternating charged moieties. These moieties could be aliphatic or aromatic.

A second mechanism that could be employed to modify the hydrophobicity or electrostatic charge of an acrylic based polymer would be to replace methyl vinyl ether described in Example 1 above with styrene, methyl methacrylate phthalic acid, trimellitic acid, vinyl acetate, or N-butyl acrylate. In addition, polymers or copolymers that incorporate coumarone, indene and carbazole could also be envisioned. These aromatic structures linked as copolymers to moieties bearing carboxylic acid, sulfonates or sulfates would add variation to the hydrophobicity and electrostatic profile of the polymer or copolymer and can be readily synthesized using standard technology (Brydson, J. A. Plastics Materials, second edition, Van Nostrand Reinhold Company, New York (1970)).

A third mechanism that one could employ to alter the hydrophobic or electrostatic nature of a copolymer as depicted in Formula II and FIG. 1 would be to modify the anhydride intermediate of the copolymer to form a half ester. To do this the anhydride ring is opened up in the presence of the alcohol intermediate of the desired moiety to be added as shown in FIG. 1. Some examples of compounds with desirable functional groups for addition to the polymer backbone are shown in Table 1.

### Example 3

### Synthesis of Cellulose-Based Polymers and Copolymers or Oligomers

For the synthesis of hydroxypropyl methylcellulose trimellitate (HPMCT), 700 grams of HMPC 2910 or 2208 is dissolved in 2100 grams of acetic acid (reagent grade) in a 5 liter kneader at 70° C. Then an appropriate amount of trimellitic anhydride (Wako Pure Chemical Industries) and 275 grams of sodium acetate (reagent grade) as a catalyst are added and the reaction is allowed to proceed at 85 to 90° C. for 5 hours. After the reactions, 1200 grams of purified water is poured into the reaction mixture, and the resultant mixture is poured into an excess amount of purified water to precipitate the polymer. The crude polymer is washed well with water and then dried to yield HPMCT. Hydroxypropyl methylcellulose acetate maleate (HPMCAM) is synthesized similarly using a mixture of acetic and maleic anhydride in place of trimellitic anhydride. Other methods can be employed to generate carboxylic acid substituted polymers of this sort.

The degree of carboxylic acid substitution is dependent upon the assay conditions used and the purity of the reactants. For example, Kokubo et al. (“Development of cellulose derivatives as novel enteric coating agents soluble at pH 3.5-4.5 and higher.” Chem. Pharm. Bull. 45:1350-1353 (1997)) demonstrate how the degree of substitution per unit of glucose of methoxyl, hydroxypropoxyl, and trimellityl can have large differences in the pH solubility of the resulting HPMCT polymer. Therefore, given the prior art, it was not obvious that simply changing the substitution from a dicarboxylic acid moiety like phthalate to a tricarboxylic acid moiety like trimellitate would yield a compound with superior solubility and carboxylilc acid group dissociation at low pH and at the same time be an effective agent against multiple infectious organisms. Just as each compound and each variant with respect to substitution per mole of glucose, needed to be tested empirically for their solubility and carboxylic acid dissociation profiles, there also was no a priori predictive indicator of how each would affect the different infectious agents described in this application.

The degree of substitution of the HPMCT polymer used in the following assay contained approximately 35 wt percent trimellitate. Given the effectiveness of HPMCT at 35% trimellitate substitution presented in this application, it is extremely likely that polymers with different percentages of carboxylic acid containing moieties would also be capable of demonstrating effective anti-viral activity.

In addition to the electrostatic enhancement provided by the trimellitate group to the cellulose backbone, the ability of the polymer to interact with viral glycoproteins is also enhanced by the presence of the phenolic ring. Specific hydrophobic forces can help stabilize the interaction of the polymers, copolymers and oligomers of this invention with HIV-1 gp120 and gp41. Therefore, striking a balance between electrostatic and hydrophobic interaction capability of the compounds of this invention is important to molecular binding of said compound with gylcoproteins on viral and/or cellular surface. Interaction with viral surface proteins including gp120 and gp 41 specifically requires both electrostatic and hydrophobic interaction to effect tight binging that would prevent viral binding cell surface receptors such CD4 or coreceptor like CCR5 and CXCR4. In order to achieve tight binding that blocks infectivity of cells the resulting polymer should be preferably present in the molecularly dispersed state. Therefore, the presence of phenyl groups as in the case of trimellitic modification is desirable for tailoring the hydrophobicity function of the molecule in order to affect the desired biological activity. According to the present invention, hydrophobicity can be imparted by selecting an intermediate anhydride, or other equivalent modifying reagent, with a strong hydrophobic group such as those bearing one or more aromatic rings including phenyl, naphthyl, and the like with known hydrophobic character. It is thus feasible to tailor the molecule with a smaller number of strong hydrophobic group like naphthyl or a larger number of less hydrophobic groups like phenyl. One skilled in the art possesses the ability to strike the above balance between hydrophobility, solubility and dissociation properties by manipulating the parameters of the modification and degree of substitution to arrive at the desired performance. The modifications according to the present invention are not limited to reactions with anhydrides but include any substitution at R or any OH groups in the cellulosic backbone skeleton. Therefore the scope of the invention should not be limited by the discrete formulae or examples covered in the specification.

To illustrate the versatility of this application Table 1 lists a partial list of moieties that could be covalently linked to a cellulose or acrylic polymer backbone, using the above described procedures, or a procedure similar to it that someone skilled in the art could realize.

TABLE 1
Substitutions for cellulose or acrylic-based oligomers, copolymers, or
polymers.

| *R | **pKa Values |
|---|---|
| 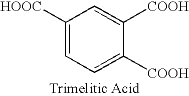 | 2.52, 3.84, 5.2 |
| 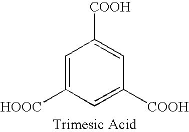 | 3.12, 3.89, 4.7 |
| 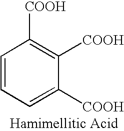 | 2.8, 4.2, 5.87 |
| 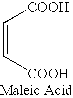 | 1.93, 6.58 |
| 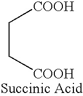 | 4.19, 5.48 |
| 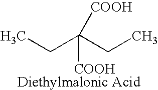 | |
| 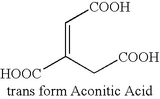 | |
| MVE/MA copolymer of | 3.51, 6.41 |
| methyl vinyl ether and | |
| maleic acid | |
| 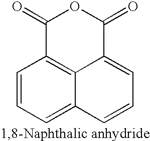 | — |
| 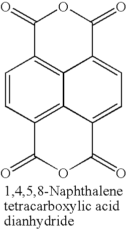 | — |
| 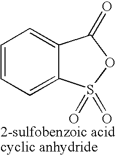 | — |
| 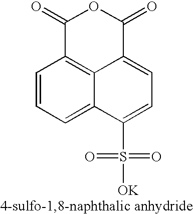 | — |
| 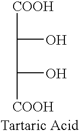 | (+)-2.99, 4.4 (−)-3.03, 4.4 Meso-3.22, 4.85 |
| 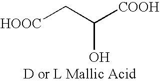 | 3.4, 5.2 |
| Vinyl acetic acid | 4.42 |

*R = moiety that when covalently attached to the polymer, copolymer, or oligomer backbone results in a molecule that is able to remain molecularly dispersed, and mostly dissociated, in solution over a wide range of pH.
**pKa values given at room temperature and taken from a variety of sources including (Hall, H. K., J. Am Chem. Soc. 79:5439-5441, 1957; Handbook of Chemistry and Physics (Hodgman, C. D., editor in Chief, Chemical Rubber Publishing Company, Cleveland, OH p. 1636-1637, 1951).

It is obvious to one skilled in synthetic organic chemistry that Table 1 represents only a partial list, and that many more examples are possible provided that no other reactive functionalities are present which would compete with the primary desired reaction of forming substituted cellulose or acrylic-based polymers or oligomers. It is also possible for one skilled in the art to find one or more active compounds in this class by performing the above synthesis or similar methods using combinatorial synthesis or equivalent schemes by altering the mono carboxylic acid moiety, or the di or tri carboxylic acid moiety, or a mixed moiety containing both carboxylic acid groups and sulfate or sulfonate groups, or a moiety containing a sulfate or sulfonate group. It is also now obvious to attempt to add additional hydrophobicity to the polymer and still retain the carboxylic acid moiety. This can be accomplished in a number of ways including the addition of a naphthalene group such as those shown in Table 1 (naphthalene tetracarboxylic dianhydride or naphthalimide) to the cellulose backbone. This type of experimentation is deemed obvious by adopting the systematic scientific method by one skilled in the art.

It is also obvious to one skilled in the art that the substitution at position R of Formula I or Formula II can be obtained by using a mixture of the compounds identified or suggested in this example. Hydroxypropyl methylcellulose acetate maleate (HPMCAM) is just such a compound in which a mixture of acetic and maleic anhydride is used to derivatize the hydroxyproply methyl cellulose backbone.

Cellulose acetate trimellitate (CAT) can be prepared by reacting the partial acetate ester of cellulose with trimellitic anhydride in the presence of a tertiary organic base such as pyridine. It is also obvious to one skilled in the art that any anhydride could be substituted for trimellitate to produce the corresponding cellulose acetate derivative.- It is also possible to produce molecules having a mixture of functional groups simply by using a mixture of different anhydrides during the synthesis procedure. For example, using methods that would produce CAP or CAT, the phthalate or trimellitate anhydride could be mixed with 2-sulfobenzoic acid cyclic anhydride in various ratios, to produce polymers or oligomers that bear both phthalate or trimellitate and 2-sulfobenzoate. The addition of 2-sulfobenzoate with phthalate would produce a polymer capable of remaining molecularly dispersed in an aqueous solution, and partially dissociated over a greater range of pH than is noted for CAP.

### Example 4

### Cellulose-Based Polymers and Copolymers or Oligomers Bearing Sulfate or Sulfonate Groups

As described in Example 3 above one mechanism that can be used to introduce sulfate or sulfonate groups onto a cellulose-based backbone is to use a moiety such as 2-sulfobenzoic acid anhydride or 4-sulfo-1,8-naphthalic anhydride. It is also obvious to one skilled in the art that the substitution at position R can be obtained by using a mixture of the moiety bearing the sulfate or sulfonate group and moieties having other constituents such as carboxylic acid groups.

Alternatively sulfation can be achieved by direct chemical linkage to the cellulosic-backbone. For example under mild conditions adducts of sulfur trioxide (SO₃) such as pyridine-sulfur trioxide in aprotic solvents is added to the cellulosic-based polymer or copolymer or oligomer which is prepared in DMF. After 1 hour at 40° C., the reaction is interrupted by the addition of 1.6 ml of water, and the raw product is precipitated with three volumes of cold ethanol saturated with anhydrous sodium acetate and then collected by centrifugation (Maruyama, T., Tioda, T, Imanari, T., Yu, G., Lindhardt, R. J., “Conformational changes and anticoagulant activity of chondroitin sulfate following its O-sulfonation.” Carbohydrate Research 306:35-43, (1998)).

### Example 5

### Cytotoxicity Analysis of HPMCT

HeLa cells (ATCC designation CCL-2) were maintained in Dulbecco's Modified Eagle's medium (DMEM). P4-CCR5 (P4R5 cells) (AIDS Reagent Program #3580) were cultured in DMEM with 0.1 ug/ml puromycin as described by Charneau et al. (“HIV-1 reverse transcription. A termination step at the center of the genome.” J. Mol. Biol. 241:651 -652 (1994)). Sup-T1 human T lymphocytes (ATCC designation CRL-1942) were cultured in RPMI 1640. All three cell types were cultured in media supplemented with 10% fetal bovine serum (FBS), L-glutamate (0.3 mg/ml), antibiotics (penicillin, streptomycin, and kanamycin at 0.04 mg/ml each), and 0.05% sodium bicarbonate.

All compounds were assessed for cytotoxicity using a standard two hour exposure of HeLa or P4-CCR5 (P4R5) target cells to the drug candidates. P4-CCR5 cells (NIH AIDS Reagent Program) are HeLa cells engineered to express CD4 and CCR5 and were utilized in experiments evaluating anti-viral activity of candidate compounds of this application. These and subsequent assessments of cell viability following exposure to candidate compounds were conducted using the MTT cell viability assay, in which cell viability is measured spectrophotometrically by conversion of MTT (3-[4,5-dimethylthiazol-2-yl]-2,5-diphenyl tetrazolium bromide) to a purple formazan product (Pauwels, R., Balzarini, J., Baba, M., Snoeck, R., Schols, D., Herdewijn, P., Desmyter, J., and De Clercq, E. “Rapid and automated tetrazolium-based colorimetric assay for the detection of anti-HIV compounds.” J Virol. Methods 20:309-321, (1988)). In typical assays P4-CCR5 cells were exposed to dextran sulfate (DS) and various cellulose or acrylic-based polymers for 2 hr at concentrations ranging from 0.00001% to 2%. Cytotoxicity evaluations between 10 min and 6 hr are usually employed because HIV-1 exposure would be most likely to occur during this time period following application of a topical microbicide.

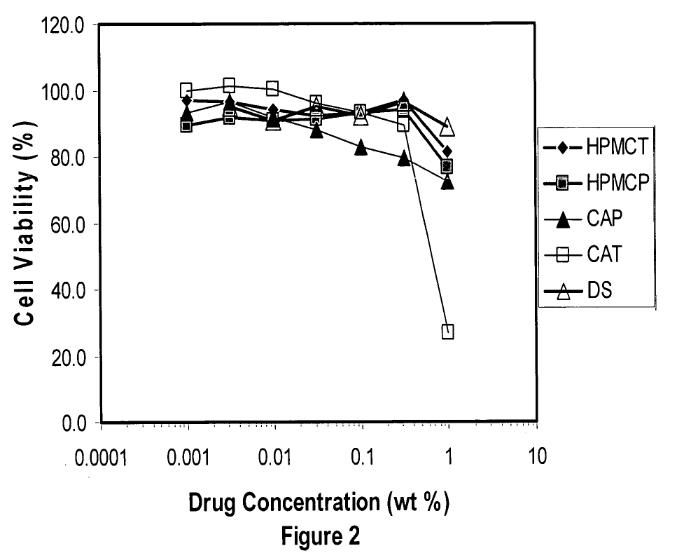

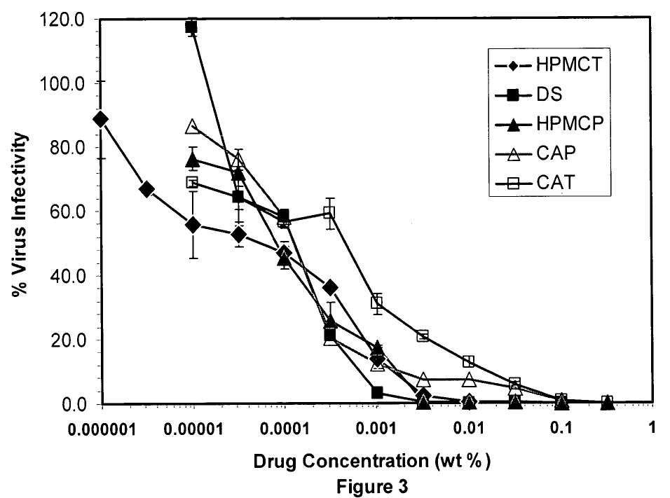

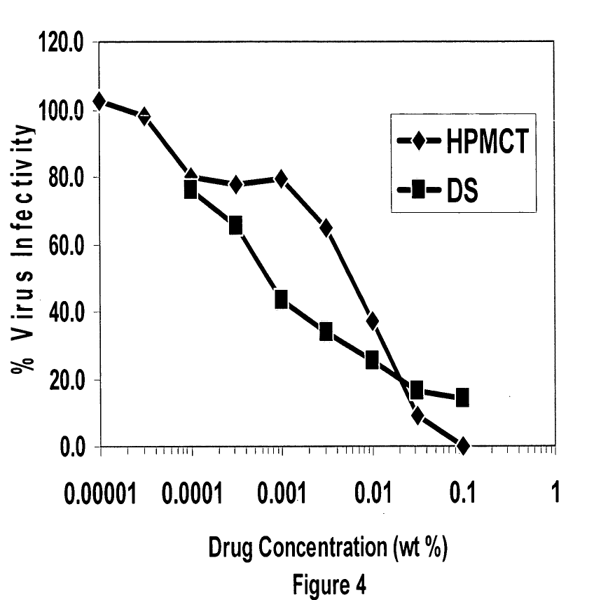

In FIG. 2 Hydroxypropyl methylcellulose-based compounds including, Hydroxypropyl methyl cellulose trimellitate (HPMCT), hydroxypropyl methylcellulose phthalate (HPMCP), and cellulose-based compounds such as cellulose acetate phthalate (CAP), and cellulose acetate trimellitate (CAT) were tested in head-to-head fashion for their effect on P4-CCR5 cell metabolism using the MTT assay described above. The concentration need to inhibit cellular metabolism by 50% (CC50) for each compound tested in this assay system is shown in Table 2. In addition the toxicity experiments were designed so that the level of exposure and the time of exposure would mimic the efficacy studies in VBI assays shown in FIGS. 3 and 4. In these experiments P4R5cells were incubated for 2 hrs in the presence of the indicated compounds after which the drug was washed off and the cells further incubated in growth media alone for an additional 48 hrs at 37° C. in a 5% CO₂ atmosphere. At this time the cells were assessed for viability by monitoring their energy production using the tetrazolium dye MTT assay as described by Rando et al. (“Suppression of human immunodeficiency virus type 1 activity in vitro by oligonucleotides which form intramolecular tetrads.” J. Biol. Chem. 270:1754-1760 (1995)). The cytotoxic concentration is many times indicated as the CC50, or concentration of compound needed to reduce cell viability by 50%. This toxicity value, when taken together with the 50% inhibitory concentration (IC50), or concentration needed to reduce cell-free HIV-1IIIB virus infectivity by 50%, is used to tabulate a therapeutic index or TI. The CC50 and IC50 used to plot the TI need to be of a similar format with respect to exposure of virus and/or cells to drug. In FIG. 2 only one compound (CAT) inhibited cell metabolism by greater than 50% at the highest concentration used. Therefore, any TI described in the text is given as a greater than value since the numerator is >1% for all compounds except CAT.

### Example 6

### In vitro Anti—HIV-1 Efficacy Experiments

a. Anti-HIV-1 Culture assays formats. In vitro detection of infectivity following exposure of virus or cells to cellulose or acrylic polymers relied primarily on the use of indicator cells that produce β-galactosidase (β-gal) as a consequence of HIV-1 infection and a chemiluminescence-based method for quantitating levels of β-gal expression. P4-R5 MAGI (multinuclear activation of galactosidase indicator) cells were used to detect both X4 and R5 strains of HIV-1 (strains that use the CXCR4 and CCR5 chemokine receptors, respectively). Although this cell line can be treated to visualize β-gal expression in subsequent cell counts, assays described within this proposal used the Tropix Galacto-Star assay system to measure β-gal production. This system facilitates the chemiluminescent detection of β-gal in cell lysates. The advantage of this system over cell staining and counting is that it is a fast and easy assay that is highly sensitive and can detect a wide range of β-gal expression. This system, combined with P4-CCR5 MAGI cells, permits sensitive, reproducible detection of infectious virus following exposure to microbicidal compounds 24 to 48 h post-infection.

Viral Binding inhibition (VBI) assays are conducted as follows. On day one, virus (X4-, R5-, or X4R5-tropic; 8 μl at approximately 10⁷ TCID₅₀ per ml) is mixed in RPMI 1640 supplemented with 10% FBS and with test compounds at concentrations decreasing in third log increments from 1%. Aliquots of this mixture are immediately placed on P4-R5 cells and incubated for 2 hr at 37° C. After 2 hr, cells are washed twice with PBS and provided with 2 ml fresh media. After 46 hr at 37° C., the cells are washed twice in PBS and lysed in the well using 125 μl lysis buffer. Activity is assessed as described above.

In cell-free virus inhibition (CFI) assays HPMCT and other cellulose-based polymers will be assessed for their ability to inactivate cell-free virus. Assays use a range of concentrations decreasing in third log increments. Briefly, 8×10⁴ P4-CCR5 cells are plated in 12-well plates 24 hr prior to the assay. On the day of the assay, 5 μl of serially diluted compound are mixed with an equal volume of virus (approximately 10⁴-10⁵ tissue culture infectious dose₅₀ (TCID₅₀) per μl) and incubated for 10 minutes at 37° C. After the incubation period, the mixture is diluted (100-fold in RPMI 1640 media including 10% FBS) and aliquots are added to duplicate wells at 450 μl per well. After a 2-hr incubation period at 37° C., an additional 2 ml of new media is added to the cells. At 46 hr post-infection at 37° C., the cells are washed twice with phosphate buffered saline (PBS) and lysed using 125 μl lysis buffer (Galacto Star). HIV-1 infectivity is measured by mixing 2-20 μl of centrifuged lysate with reaction buffer (Galacto Star), incubating the mixture for 1 hr at RT, and quantitating the subsequent luminescence.

Similar experimental protocols can be utilized for microbicidal treatment of infected cell lines (cell associated virus inhibition (CAI) assays). For example, SupT1 cells (3×10⁶) are infected with HIV-1 IIIB (30 μl of a 1:10 dilution of virus stock) in RPMI media (30 μl) and incubated for 48 hr. Infected SupT1 cells are pelleted and resuspended (8×10⁵ cells/ml). Microbicides (5 μl) are added to infected SupT1 cells (95 μl) and incubated (10 min at 37° C.). After incubation, the cell and microbicide mixture will be diluted in RPMI media (1:10) and 300 μl will be added to the appropriate wells in triplicate. In the wells, target P4-R5 cells will be present. Production of infectious virus will result in β-gal induction in the P4-R5 targets. Plates are incubated (2 hr at 37° C.), washed (2×) with PBS and then media (2 ml) is added and before further incubation (22-46 hr). Cells are then aspirated and washed (2×) and then incubated (10 min at room temperature) with lysis buffer (125 μl). Cell lysates are assayed utilizing the Galacto-Star™ kit (Tropix, Bedford, Mass.).

In FIG. 3 and Table 2 is presented the dose response curves and IC50 values for DS, HPMCT, HPMCP, CAT and CAP when used to inhibit HIV-1IIIB in the VBI assay. The IC50 value is the concentration of drug needed to inhibit virus infectivity by 50%. The results from these experiments show that all compounds were effective inhibitors of HIV-1 in this assay system and fairly similar in their overall activity with the difference between calculated IC50s for the most (HPMCT IC50=0.00009%) and least (CAT IC50=0.0005%) active cellulose-based compound less then a factor of 10 (see Table 2).

In FIG. 4 and Table 2 the dose response curve and IC50 value showing the effect of HPMCT on HIV-1BaL in the VBI assay is shown. It is interesting to note that the overall activity against HIV-1BaL is approximately 10-fold lower than that observed against the CXCR4 tropic strain of virus for both HPMCT and DS.

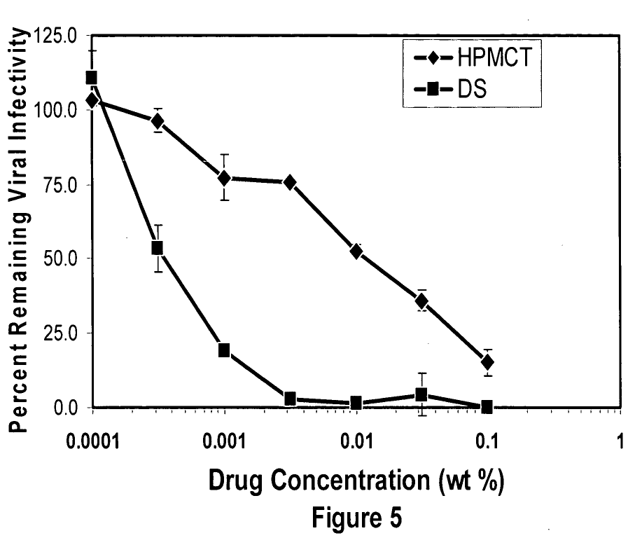

In FIG. 5 and Table 2 the dose response curve and IC50 value the effect of HPMCT on HIV-1IIIB in a cell free virus inhibition (CFI) assay is shown. While HPMCT still displays potent activity, it is not as effective in this assay as in the VBI assay while the control drug DS has a level of activity similar to what it displayed in the VBI assay. The reported mechanism of action for CAP (Neurath, A. R., et al. “Anti-HIV-1 activity of cellulose acetate phthalate: Synergy with soluble CD4 and induction of “dead-end” gp41 six-helix bundles.” BMC Infectious Diseases 2:6 (2002); Manson, K. H. Wyand, M. S., Miller, C., and Neurath, A. R. “The effect of a cellulose acetate phthalate topical cream on vaginal transmission of simian immunodeficiency virus in rhesus monkeys.” Antimicrob. Agents Chemother 44:3199-3202 (2000); Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120.” BMC Infectious Diseases 1:17 (2001)), is via interfering with the co-receptor interactions on the cell surface with the viral gp120. This activity may occur after gp120 has undergone a conformational change post binding with the main cellular receptor CD4. Therefore in this short exposure to HPMCT the co-receptor binding surface of gp120 may not be accessible to the cellulose polymer. The mechanism of action for DS is known to be via direct interaction with the V3 loop of HIV-1 gp120 (Esté, J. A., Schols, D., De Vreese, K., Cherepanov, P., Witvrouw, M., Pannecouque, C., Debyser, Z., Desmyter, J., Rando, R. F., and De Clercq, E., “Human immunodeficiency virus glycoprotein gp120 as the primary target for the antiviral action of AR177 (Zintevir).” Mol. Pharm. 53:340-345 (1998)). By binding to the V3 loop of the viral glycoprotein DS interferes with gp120-CD4 interactions. Therefore DS maintains its potency in the short CFI assay duration because it binds to the exposed V3 loop of gp120 and prevents the virus from contacting CD4 in the subsequent steps in the assay. In contrast HPMCT binds to portions of the viral glycoprotein that are generally exposed after the virus binds to the cell (gp120-CD4) and therefore, in the CFI assay system most of the HPMCT is diluted out of the system before the virus is exposed to target cells.

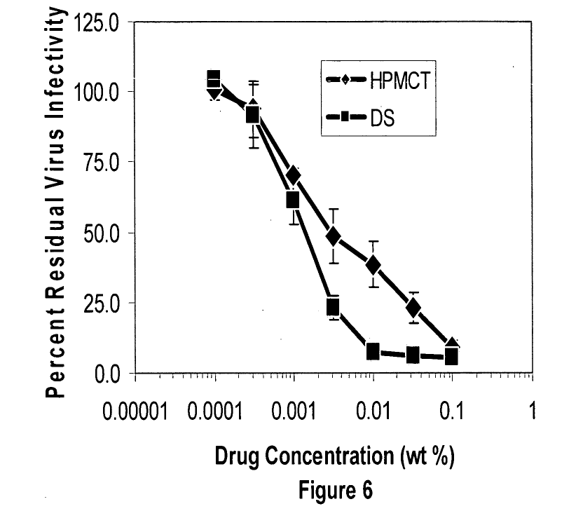

FIG. 6 and Table 2 shows the dose response curve and IC50 value for HPMCT's effect on HIV-1IIIB using a cell associated virus inhibition (CAI) assay. In this assay cell associated virus was incubated with HPMCT or DS for 10 minutes before dilution an exposure to uninfected reporter cells for 2 hrs. Reporter cells where then washed to remove drug and residual virus in the culture media and then incubate for an additional 48 hrs at 37° C. in a 5% CO₂ atmosphere. The data in this experiment shows that HPMCT is much more effective at inhibiting virus transmission than in the CFI assay. In this assay it is possible for CD4 interactions with gp120 to occur before drug is removed from the culture media thereby giving HPMCT access to exposed surfaces of gp120 that form the basis of interaction with the cellular co-receptors CXCR4 or CCR5.

The acrylic copolymer MVE/MA was also tested for its effect on HIV-1IIIB in a VBI assay. MVE/MA is commercially available in a variety of different molecular size ranges. In these studies we used low molecular weight MVE/MA having an average mol. wt. in the range of 216,000 daltons, and high molecular weight MVE/MA which had an average molecular weight in the range of 1.98×10⁶ daltons. MVE/MA was added to P4-CCR5 cells in culture in the presence of virus for 2 hrs. The cells were then washed three times with fresh medium and then further incubated for 48 hr at 37° C. in a 5% CO₂ atmosphere before the level of β-gal production was monitored. The results from this experiment are shown in Table 2. It is clear that MVE/MA itself is not toxic to cells following a 2 hr exposure at concentrations above 0.1%, while its IC50 against HIV-1IIIB in the VBI was determined to be 2.3 μg/ml (low molecule weight MVE/MA), and 2.8 μg/mi for the high molecular weight species which corresponds to 0.00023 and 0.00028 percent respectively.

TABLE 2
Effect of polymers on HIV-1 transmission.

| | IC50 | | CC50 |
|---|---|---|---|
| Assay System | (wt. %) | TI | (wt. %)** |
| VBI | | | |
| DS | 0.00015 | >10000 | >1 |
| HPMCT | 0.00009 | >11000 | >1 |
| HPMCP | 0.0006 | >1600 | >1 |
| CAP | 0.00015 | >10000 | >1 |
| CAT | 0.00054 | 1296 | 0.7 |
| MVE/MA acrylic copolymer | 0.00023 | 891 | 0.205 |
| 216 K mol. wt. fraction | | | |
| MVE/MA acrylic copolymer | 0.00028 | 678 | 0.19 |
| 1.98 MM mol. wt. fraction | | | |
| CFI* | | | |
| DS | 0.0004 | >2500 | >1 |
| HPMCT | 0.01 | >100 | >1 |
| CAI* | | | |
| DS | 0.002 | >500 | >1 |
| HPMCT | 0.003 | >300 | >1 |

*CFI, and CAI assays used a ten minute incubation of drug with virus before dilution and addition of virus to cells.
**CC50s were calculated using an MTT assay to assess cell viability 48 hrs after cells had been exposed to test compound for 2 hr.

b. Anti-HIV-1 efficacy of HPMCT in combination with the cationic polybiguanide PEHMB. The paradigm for effective HIV-1 therapy (for systemic infections) is the use of combination drug regimens. Combination therapy has proven effective at reducing viremia, delaying the onset of AIDS, and retarding the emergence of drug-resistant virus. At this time the most effective microbicide regimen has not been established. It may be that in order to block sexual transmission of HIV-1 several drugs, having different mechanisms of action will need to be applied in the same formulation. Therefore, our strategy for augmenting or broadening the spectrum of HPMCT activity is to combine it with other compounds that have different mechanisms of action against HIV-1. As an example, we have investigated the use of polyethylene hexamethylene biguanide or PEHMB (Catalone, B. J., et al. “Mouse model of cervicovaginal toxicity and inflammation for the preclinical evaluation of topical vaginal microbicides.” Antimicrob. Agents and Chemother. 2004 in press) combined with HPMCT. PEHMB is a cationic polymer made up of alternating ethylene and hexamethylene around a biguanide core. In these assays a 1.0% wt/vol stock solutions of HPMCT dissolved in 20 mM sodium citrate buffer pH 5.0, and a 5% PEHMB wt/vol solution made up in saline were used.

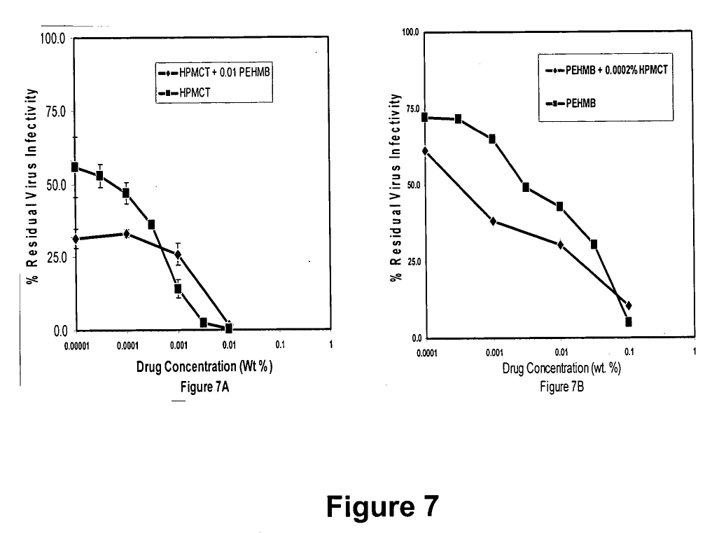

In vitro cytotoxicity experiments demonstrated that combinations of PEHMB and HPMCT, in which the concentration of one component was varied while the other was kept constant, were non-cytotoxic after a two hour exposure of compounds to test cells, at the concentrations tested as was the case for PEHMB and HPMCT tested alone (FIG. 2). Then using a VBI assay and HIV-1 strain IIIB, HPMCT was equally or more effective when 0.01% PEHMB was combined with various concentrations of HPMCT than when using HPMCT alone (FIG. 7A). Similar results were observed when the concentration of HPMCT was held constant at 0.0002% and the concentration of PEHMB was varied (FIG. 7B). These data show that a negatively charged agent can be successfully combined with a positively charged agent and when used in such combinations can help reduce the level of virus infectivity below that which would be predicted by simple addition of their effectiveness.

While logically it appears that negatively-charged polymers like HPMCT would be a poor choice for inclusion in a combination with the positively charged PEHMB, we believe that the antiviral activity of PEHMB, and PEHMB-derived molecules, relies not only upon their positive charge, but also upon their three-dimensional shape. Therefore it may be possible to obtain mixtures of polyanionic compounds with PEHMB at defined ratios which allow for the full expression of the antiviral properties of the individual components without exhibiting any deleterious effects due to their mixing. As seen in FIG. 6, at least within the concentration ranges of PEHMB and HPMCT tested no antagonistic effects are observed when these two molecules were combined. These data strongly suggest that HPMCT can be used in combination with other agents producing at least additive effects, and it is possible under the appropriate conditions to mix low cost polymers with completely different chemical features.

### Example 7

### Effect of HPMCT on Herpes Simplex Virus Infections

Herpes simplex virus plaque reduction assays were performed as described by Fennewald et al. (“Inhibition of Herpes Simplex Virus in culture by oligonucleotides composed entirely of deoxyguanosine and thymnidine.” Antiviral Research 26:37-54 (1995)). This assay was a variation on the cytopathic effect assay described by Ehrlich et al. (Ehrlich, J., Sloan, B. J., Miller, F. A., and Machamer, H. E., “Searching for antiviral materials from microbial fermentations.” Ann N.Y. Acad. Sci 130:5-16 (1965)). Basically cells such as Vero or CV-1 cells are seeded onto a 96-well culture plate at approximately 1×10⁴ cells/well in 0.1 ml of minimal essential medium with Earle salts supplemented with 10% heat inactivated fetal bovine serum (FBS) and pennstrep (100 U/ml penicillin G, 100 ug/ml streptomycin) and incubated at 37° C. in a 5% CO₂ atmosphere overnight. The medium was then removed and 50 ul of medium containing 30-50 plaque forming units (PFU) of HSV1 or HSV2, diluted in test medium and various concentrations of test compound are added to the wells. The starting material for this assay was a 0.6% wt/vol stock solutions of HPMCT dissolved in 20 mM sodium citrate buffer pH 5.0. Test medium consists of MEM supplemented with 2% FBS and pennstrep. The virus was allowed to adsorb to the cells, in the presence of test compound, for 60 min at 37° C. The test medium is then removed and the cells are rinsed 3 times with fresh medium. A final 100 ul of test medium is added to the cells and the plates are returned to 37° C. Cytopathic effects are scored 40-48 hr post infection when control wells (no drug) showed maximum cytopathic effect.

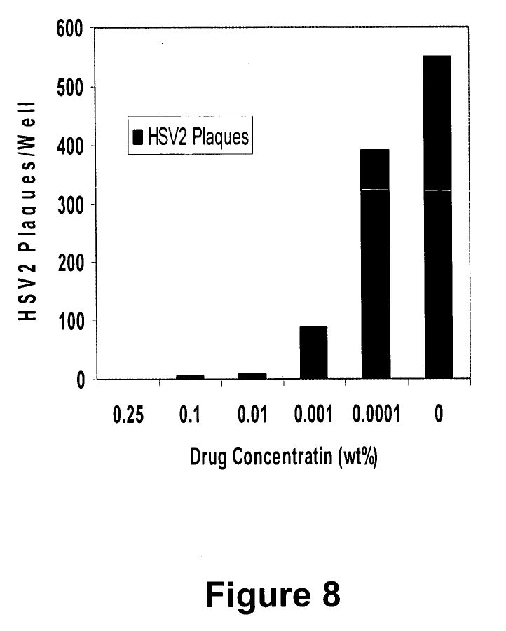

In these experiments HPMCT was added to HSV2 stock for ten minutes before the mixture was added to cells for 60 min as described above. Forty to 48 hrs post removal of drug from the culture media the control wells that received no drug treatment had over 500 plaques per well. Wells treated with 0.0001% HPMCT for the indicated amount of time had less than 400 plaques per well while wells treated with 0.25% HPMCT had no visible plaques, the IC50 for HPMCT in this assay system was below 0.001% (FIG. 8). This result demonstrates the potency of HPMCT as ani anti-herpes simplex virus agent.

### Example 8

### Effect of HPMCT on Bacterial Pathogens

To test the effect of HPMCT on bacterial pathogens the cellulosic-based polymer was dissolved in 20 mM sodium citrate buffer pH 5.0 (0.6% final concentration of stock solution) and then mixed in equal parts with bacterial suspensions as described. First bacteria are sub-cultured 1-2 days prior to the assay by streaking cultures onto suitable agar plates such as Trypticase soy agar. Aseptic technique is used in all aspects of this protocol. A fresh bacterial colony is then used to inoculate 15 ml of 2× culture medium. To the first nine (9) columns of a 96 well plate, 100 μl of the inoculated 2× culture broth is transferred into the wells using a multi channel pipette. The remaining three (3) columns (usually numbered 10-12) are used as a sterility control. To these columns, 100 μl of sterile 2× culture broth is added to each well. The culture medium in the second through eighth rows (usually designated B—H) is diluted by the addition of 80 μl of sterile water to those wells. The volume in wells B through H is at this time 180 μl. The antimicrobial solutions are diluted with water to twice the desired concentration of the uppermost starting concentration. For instance, if the highest test concentration is 1%, the solution should be prepared at 2%. For some compounds, no dilution may be needed. To the first row (usually designated as “A”), 100 μl of 2× test solution is added to each well. The solution is thoroughly mixed by re-pipetting five times. The total volume of the well is now 200 μl. A 1:10 serial dilution is now performed from Row A through Row G by transferring 20 μl from the higher concentration to the subsequent row using a multi channel pipette. This results in a six log reduction in the concentration of the test compound. In Row G, 20 μl is removed and discarded. No test compound is added to Row H (positive control for growth). The 96 well plate is placed on a shaker in an incubator with the temperature set for the organism of choice (usually 30° C. or 37° C.). After 24 hours, the optical density of the cultures is measured on a 96 well plate reader. Row H serves as a positive control for growth. Columns 10 through 12 serve as negative controls and as a measurement of the optical density of the test solution at different concentrations. The test solution were considered effective at a given concentration if the optical density of the inoculated wells is statistically the same as the negative control wells.

The above described HPMCT formulation was tested for its inactivating effect on the following bacterial pathogens Pseudomonas aeruginosa and Escherichia Coli. Both strains were cultured in Minimal Culture Medium (M9 medium). The results shown in Table 3 indicate that both bacterial strains lost the capacity to replicate after exposure to HPMCT. Vantocil (polyhexamethylene biguanide) is a commercially available disinfectant and was used as a positive control in these experiments. PEHMB is a variant of Vantocil and was also used as a control in these experiments. The activity of HPMCT against the indicated species would strongly suggest that the compound will be active against a variety of bacterial strains including but not limited to Trichomonas vaginalis, Neisseris gonorrhoeae Haemopholus ducreyi, or Chlamydia trachomatis, Gardnerella vaginalis, Mycoplasma hominis, Mycoplasma capricolum, Mobiluncus curtisi, Prevotella corporis, Calymmatobacterium granulomatis, and Treponema pallidum. Pseudomonas aeruginosa, Streptococcus gordonii, or S. oralis for dental plaque, Actinomyces spp, and Veillonella spp.

TABLE 3
Minimum Inhibitory Concentration for
HPMCT against two bacterial strains.

| | | |
|---|---|---|
| Vantocil* | PEHMB* | HPMCT* |

| | |
|---|---|
| Bacterial strain | MIC (wt. %) |

| | | | |
|---|---|---|---|
| Escherichia coli | 0.06 | 0.125 | 0.31 |
| Pseudomonas aeruginosa | 0.06 | 0.5 | 0.16 |

*Vantocil is polyhexamethylene biguanide, PEHMB is a variant of Vantocil, and HPCMT is hydroxypropyl methylcellulose trimellitate.

### Example 9

### Effect of pH on Solubility of Cellulose-Based Polymers.

Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)) demonstrated that by careful selection of carboxylic acid containing moieties used to link with a cellulosic polymer backbone, the overall pKa of the cellulosic-based polymer could be modified. In addition in 2000 Neurath reported that CAP and HMPCP are effective agents against sexually transmitted diseases (Neurath A. R. et al. “Methods and compositions for decreasing the frequency of HIV, herpsevirus and sexually transmitted bacterial infections.” U.S. Pat. No. 6,165,493, 2000). In this study Neurath's group appreciates the fact that carboxylic acid groups of CAP and HPMCP are not entirely dissociated at the vaginal pH and actually propose to use micron size particulate formulations of their identified compounds to help get around the solubility issue (Neurath A. R. et al. U.S. Pat. No. 6,165,493 (2000); Manson, K. H. et al. “Effect of a Cellulose Acetate Phthalate Topical Cream on Vaginal Transmission of Simian Immunodeficency Virus in Rhesus Monkeys,” Antimicrobial Agents and Chemotherapy 44:3199-3202 (2000)). Therefore, the use of chemical moieties to enhance the low pH solubility and significant dissociation of the ionizable functional groups of cellulosic-based, or other, polymers and then using those polymers as anti-infective agents would be extremely helpful to the overall anti-infective properties of a microbicide. Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)) demonstrate using dissolution time versus pH curves the solubility of compounds such as HPMCT and hydroxypropyl methylcellulose acetate maleate (HPMCAM) in low pH solutions (dissolution pH for these two compounds was determined to be between 3.5 and 4.5) and compared these measured values with historical data on the dissolution pH of CAP (pH 6.2) and HPMCP (pH ˜5.0 to 5.5. These data are consistent with the pKa reported for second carboxylic acid group on trimellitate (3.84) and phthalate (5.28).

The toxicity and efficacy assays described in Examples 5-7 are routinely performed in eukaryotic cell culture media that is buffered and maintains a pH in the neutral range throughout the time course of the experiment. In these examples the IC50s and CC50s of the four cellulose based polymers tested (HPMCT, CAT, HPMCP and CAP) were roughly equivalent. However, to illustrate the point that the trimellitate bearing compounds could be differentiated from, and therefore superior to, the phthalate bearing compounds, we performed a simple experiment to show that only HPMCT and CAT were able to remain molecularly dispersed and mostly dissociated over the range of pH encountered in the vaginal lumen. This experiment also confirmed the pH dissolution data reported by Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)).

In this experiment 1% solutions of HPMCT, CAP, CAT and HPMCP (all dissolved in 100 mM Na citrate pH 6.0) were exposed in a drop wise fashion to 0.5N HCl. After each small aliquot of added HCl the samples were vortexed, allowed to settle, and then observed for clarity and the pH was measured. The results from this mostly qualitative experiment are presented in Table 4. It is readily observed that the solutions containing the trimelliate moiety remained clear at much lower pH values than those containing the phthalate group. In addition, at lower pH HPMCT and CAT did not ‘gel’ to the same extent indicating that more material remains molecularly dispersed over this range of pH.

TABLE 4
Titration of HCl into 1% solutions of cellulose based polymers.

| | Visual Solution Characteristics at Selected pH | | | | | | | | | |
|---|---|---|---|---|---|---|---|---|---|---|
| Compound | 5.75 | 5.5 | 5.25 | 5.0 | 4.75 | 4.5 | 4.25 | 4.0 | 3.75 | 3.5 |
| CAP | Clear | Clear | Clear | Cloudy | viscous | Thick | — | — | — | — |
| | | | | | cloudy | gelled | | | | |
| | | | | | soln | mass | | | | |
| HPMCP | Clear | Clear | Clear | Cloudy | viscous | viscous | Total | — | — | — |
| | | | | | cloudy | cloudy | gelled | | | |
| | | | | | soln | soln | mass | | | |
| CAT | Clear | Clear | Clear | Clear | Clear | Clear | Viscous | Globular | — | — |
| | | | | | | | cloudy | masses | | |
| | | | | | | | soln | cloudy | | |
| HPMCT | Clear | Clear | Clear | Clear | Clear | Clear | Clear | Viscous | Viscous | Partially |
| | | | | | | | | | cloudy | gelled |

HPCMT is hydroxypropyl methyl cellulose trimellitate,
HPMCP is hydroxypropyl methyl cellulose phthalate,
CAP is cellulose acetate phthalate, and
CAT is cellulose acetate trimellitate.

### Example 10

### Drug Combination Therapy Regimens

At present combination therapy comprising at least three anti—HIV drugs has become the standard systemic treatment for HIV infected patients. This treatment paradigm was brought about by necessity in that mono- and even di-drug therapy proved ineffective at slowing the progression from HIV-1 infection to full blown AIDS. Therefore it is also likely that in the development and application of a topical agent to prevent the transmission of STDs a combination of drugs each having a different or complementary mechanism of action can be envisioned.

The methodology used in the identification of potential combinations for use against HIV-1 has been reported numerous times in the identification and development of anti-HIV-1 drugs for systemic applications (Bédard, J., May, S., Stefanac, T., Chan, L., Stamminger, T., Tyms, S., L'Heureux, L., Drach, J., Sidwell, R., and Rando, R. F. “Antiviral properties of a series of 1,6-naphthyridine and dihydroisoquinoline derivatives exhibiting potent activity against human cytomegalovirus.” Antimicrobial Agents and Chemotherapy. 44:929-937, (2000); Taylor, D., Ahmed, P., Tyms, S., Wood, L., Kelly, L., Chambers, P., Clarke, J., Bedard, J., Bowlin, T., and Rando, R. “Drug resistance and drug combination features of the human immunodeficiency virus inhibitor, BCH-10652 [(±)-2′ deoxy-3′ oxa-4′ thiocytidine, dOTC].” Antimicrobial Chemistry and Chemotherapy 11:291-301, (2000); deMuys, J. M., Gourdeau, H., Nguyen-Ba, N., Taylor, D. L., Ahmed, P. S., Mansour, T., Locas, C., Richard, N., Wainberg, M. A., and Rando, R. F. “Anti-HIV-1 activity, intracellular metabolism and pharmacokinetic evaluation of dOTC (2′-deoxy-3′-oxa-4′-thiocytidine).” Antimicrobial Agents and Chemotherapy 43:1835-1844, (1999); Gu, Z., Wainberg, M. A., Nguyen-Ba, P. L'Heureux, L., de Muys, J. -M., and Rando, R. F., “Mechanism of action and in vitro activity of 1′,3′-dioxolanylpurine nucleoside analogues against sensitive and drug-resistant human immunodeficiency virus type 1 variants.” Antimicrobial Agents and Chemotherapy 43:2376-2382, (1999)). In all cases it is of utmost importance to use one or more methods of statistical analysis of the data to discern the degree of synergy, antagonism or strictly additive effects (Chou, T. -C, and P. Talalay “Quantitative analysis of dose-effect relationships: the combined effects of multiple drugs or enzyme inhibitors.” Adv. Enzyme Regul. 22:27-55, (1984); Prichard, M. N., and C. Shipman “A Three-Dimensional Model to Analyze Drug-Drug Interactions.” Antiviral Research 14:181-206., (1990)).

It is also most likely that one will obtain optimal effects on the transmission of HIV when two or more component drugs used in combination each have a unique mechanism of action. This last statement is exemplified in FIG. 7 in which HPMCT was used in combination with the cationic polymer PEHMB. While logically it appears that the negatively-charged polymers like HPMCT or polysulfonates would be a poor choice for inclusion with a cationic compound such as PEHMB (polyethylene hexamethylene biguanide), we believe that the antiviral activity of PEHMB, and PEHMB-derived molecules, will rely not only upon their charge, but also upon their three-dimensional shape. Therefore it may be possible to obtain mixtures of polyanionic compounds with PEHMB at defined ratios, as seen in FIG. 7. A simple observation of a solution containing 0.25% PEHMB and 0.25% HPMCT in 50 mM Na Citrate pH 6.0 did not detect any undo viscosity, cloudiness or precipitation in the solution indicating that the positive and negative charged species did not interact in a fashion that would cause dissolution (not shown). Further the antiviral activity shown in FIG. 7 determined that the biologic activity of the species was not dampened in any fashion when the two drugs were added simultaneously to the reaction mixture.

It is also possible to mix two or more different negatively charged polymers, copolymers or oilgomers together in solution. The utility of this strategy is pronounced when the mechanisms of action of the ingredients are different such as would be the case if HPMCT was added together with a polysulfonated compound such as DS. Cellulosic-based compounds like CAP have been reported to interfere with virus fusion to target cells by blocking co-receptor recognition of the virus while DS is known to directly block virus attachment to cells via its primary receptor CD4. It is extremely likely that HPMCT and CAT have a mechanism of action similar to CAP.

The experimental design for most combination studies is roughly similar in that for each set of two compounds the concentration of one compound is held constant at various concentrations (e.g. the compounds IC25, IC50, IC75 or IC90 value) while the second compound is added to the reaction over a complete range of doses. Then the experiment is also performed in reversed so that the first compound is tested over a complete dose range while the second compound is held steady at one of several concentrations. Therefore the combination studies are performed using a checker board type cross pattern of drug concentrations.

Since various classes of chemical agent are being proposed as effective topical therapies for STDs that could not be utilized in systemic therapeutic applications, and these agents could be used effectively with existing systemic therapies for HIV-1, the number of potential combination permutations that could be used for topical applications is greater than that for systemic regimens. For example, as stated above HPMCT polymers could be used with cationic polymers or oligomers such as PEHMB, with other anionic compounds that have been tried (and failed) clinical trials for systemic applications such as DS, with surfactants such as SDS, or N-9, with known antibiotics, and with the different classes of drugs that have already been approved for systemic treatment of HIV-1. Some examples of the different classes of drugs available or under study are listed in Table 5. All of these examples could be used in combination with the cellulose or acrylic-based polymers, copolymers or oligomers of this current invention.

TABLE 5
Classes of agents approved or under consideration
for use in human therapy.

| Drug Class | Mechanism of Action | Drug or drug class |
|---|---|---|
| Virus | | |
| Nucleoside RT | HIV-1 RT Chain | 3TC, Tenofovir, etc. |
| Inhibitor | Termination | |
| Non Nucleoside RT | RT enzyme inhibition | UC781, CSIC, EFV^{§} |
| Inhibitor | | |
| DNA pol inhibitors | Viral DNA polymers | Acyclovir, Ganciclovir, |
| (herpesviruses) | | Cidofovir, etc. |
| Protease Inhibitor | Protease inhibition | Saquinavir, etc. |
| Fusion Inhibitor | Gp41 trimer formation | T20, CAP, HPMCT, CAT |
| HIV-1 | | |
| Fusion Inhibitor | | HPMCT, CAP |
| HSV | | |
| Binding/Fusion | CXCR4 or CCR5 | T22, AMD3100 |
| Inhibitor | co receptor | |
| | binding inhibitior | |
| Polymers, | Binding or fusion | MVE/MA, Carageenan, |
| copolymers or | inhibition | DS, sulfated dendrimers, |
| oligomers | | AR177^{†}, HPMCT, CAT, |
| (anionic) | | CAP, HPMCP |
| Polymers, | — | PEHMB and its variant |
| copolymers or | | polybiguanides* |
| oligomers | | |
| (cationic) | | |
| | HIV-1 Integrase | |
| others | | e.g. Ribavirin, |
| | | interferon |
| Bacterial | | |
| β-lactams | Peptidoglycan cell | Penicillins and |
| | wall synthesis | cephalosporins |
| tetracyclins | | |
| Aminoglycosides | Bacterial ribosomes/ | Streptomycin and |
| | translation | variations |
| macrolides | Bacterial ribosomes/ | Erythromycin and |
| | translation | variations |
| Fungal | | |
| Polyenes | Disrupt fungal | Amphotericin B, |
| | cell wall causing | Nystatin |
| | electrolyte leakage | |
| Azoles | Inhibit ergosterol | Fluconazole, |
| | biosynthesis by | Ketoconazole |
| | blocking | |
| | 14-alpha-demethylase | |
| Allylames | Disrupt ergosteral | Terbinafine |
| | synthesis | |
| Antimetabolies | Substrate for fungal | flucytosine |
| | DNA polymerase | |
| Glucan synthesis | Glucan is a key | caspofungin |
| Inhibitors | component in | |
| | fungal cell wall | |

^{†}AR177 is an effective blocker of virus binding and entry (Este J. A., et al. Mol Pharmacol.; 53(2): 340-5, 1998.
^{§}Motakis, D., and M. A. Parniak “A tight binding mode of inhibition is essential for anti-human immunodeficiency virus type 1 virucidal activity of nonnucleoside reverse transcriptase inhibitors”. Antimicrobial Agents and Chemotherapy 46: 1851-1856, 2002.
*Catalone et al. “Mouse model of cervicovaginal toxicity and inflammation for preclinical evaluation of topical vaginal microbicides.” Antimicrobial Agents. Chemotherapy vol 48, 2004.

1. A method for treating, or decreasing the frequency of transmission of a virus, or bacterial, or fungal infection in a host comprising administering to the host a therapeutically effective amount of at least one compound according to Formula I either alone or in combination with a pharmaceutically acceptable carrier, emulsifier, salt, or diluent, or other pharmaceutically active agent:
Wherein:
The cellulose backbone is substituted with one or more organic moieties such that the resultant compound is anionic in nature, molecularly dispersed and mostly dissociated in an aqueous solution over a wide range of pH (preferably from 14 to below 3.5).

2. A method according to claim 1 wherein the cellulose backbone of the composition of claim 1 is further modified by direct substitution with sulfate or sulfonate, or both, groups at one or more hydroxyl moiety on the cellulose backbone.

3. A method according to claim 1 wherein the substitution at position R is an organic hydrophobic moiety such as phenol or naphthyl, or the like.

4. A method according to claim 3 wherein the hydrophobic moiety further contains one or more anionic functional group such as a carboxylic, sulfate, or sulfonate group.

5. A method according to claim 2 wherein the cellulose based polymer CAP is further derivatized using sulfate and/or sulfonate groups covalently attached to one or more hydroxyl group on the cellulose backbone.

6. A method according to claim 2 wherein the cellulose based polymer HPMCP is further derivatized using sulfate and/or sulfonate groups covalently attached to one or more hydroxyl group on the cellulose backbone.

7. A method for treating, or decreasing the frequency of transmission of a virus, or bacterial infection in a host comprising administering to the host a therapeutically effective amount of at least one compound according to Formula I either alone or in combination with a pharmaceutically acceptable carrier, emulsifier, salt, or diluent, or other pharmaceutically active agent wherein the therapeutic agent is hydroxypropyl methylcellulose trimellitate (HPMCT).

8. A method according to claim 7 wherein the degree of trimellitate substitution to the cellulose backbone is in the range of 0.25 to 0.7 trimellityl units to each glucose unit in the backbone.

9. A method according to claim 7 wherein the overall molecular weight of the molecule can range from 500 daltons to >1.5 MM daltons.

10. A method according to claim 8 wherein the modified cellulose backbone is further substituted at one or more hydroxyl group with a sulfate or sulfonate bearing moiety.

11. A method according to claim 1 wherein the therapeutic agent is hydroxypropyl methylcellulose acetate maleate (HPMCAM).

12. A method according to claim 11 wherein the degree of substitution to the cellulose backbone is in the range of 0.15 to 0.6 maleyl units, and 0.3 to 0.7 acetyl units to each glucose unit in the backbone.

13. A method according to claim 11 wherein the overall molecular weight of the molecule can range from 500 daltons to >1.5 MM daltons.

14. A method according to claim 12 wherein the modified cellulose backbone is further substituted at one or more hydroxyl group with a sulfate or sulfonate bearing moiety.

15. A method according to claim 1 wherein the therapeutic agent is cellulose acetate trimellitate (CAT).

16. A method according to claim 15 wherein the degree of trimellitate substitution to the cellulose backbone is in the range of 0.25 to 0.7 trimellityl units to each glucose unit in the backbone.

17. A method according to claim 15 wherein the overall molecular weight of the molecule can range from 500 daltons to >1.5 MM daltons.

18. A method according to claim 16 wherein the modified cellulose backbone is further substituted at one or more hydroxyl group with a sulfate or sulfonate bearing moiety.

19. A method for treating, or decreasing the frequency of transmission of a virus, or bacterial infection in a host comprising administering to the host a therapeutically effective amount of at least one compound according to Formula I in combination with other anionic polymers, copolymers, or oligomers.

20. A method according to claim 19 wherein the combination includes HPMCT and CAT.

21. A method according to claim 19 wherein the combination includes HPMCT and HPMCAM.

22. A method according to claim 19 wherein the combination includes HPMCT and one or more sulfonated polymers, copolymers, or oligomers.

23. A method according to claim 19 wherein the combination includes HPMCT and one or more sulfated polymers, copolymers, or oligomers.

24. A method according to claim 19 wherein the combination includes HPMCT and one or more acrylic based polymers, copolymers, or oligomers.

25. A method according to claim 19 wherein the combination includes HPMCT and MVEIMA.

26. A method according to claim 19 wherein the combination includes HPMCT and a derivative of CAP in which hydroxyl groups on the cellulose backbone of CAP have been further substituted with sulfate or sulfonate bearing moieties.

27. A method according to claim 19 wherein the combination includes HPMCT and a derivative of HPMCP in which hydroxyl groups on the cellulose backbone of HPMCP have been further substituted with sulfate or sulfonate bearing moieties.

28. A method according to claim 19 wherein the combination includes HPMCT and cationic polymers, copolymers, or oligomers.

29. A method according to claim 19 wherein the combination includes CAT and HPMCAM.

30. A method according to claim 19 wherein the combination includes CAT and one or more sulfonated polymer, copolymer, or oligomer.

31. A method according to claim 19 wherein the combination includes CAT and one or more sulfated polymer, copolymer, or oligomer.

32. A method according to claim 19 wherein the combination includes CAT and one or more acrylic based polymers, copolymers, or oligomers.

33. A method according to claim 19 wherein CAT is used in combination with MVE/MA.

34. A method according to claim 19 wherein the combination includes CAT and a derivative of CAP in which hydroxyl groups on the cellulose backbone of CAP have been further substituted with sulfate or sulfonate bearing moieties.

35. A method according to claim 19 wherein the combination includes CAT and a derivative of HPMCP in which hydroxyl groups o
n the cellulose backbone of HPMCP have been further substituted with sulfate or sulfonate bearing moieties.

36. A method according to claim 15 wherein the combination includes CAT and cationic polymers, copolymers, or oligomers.

37. A method according to claim 19 wherein the combination includes HPMCAM and one or more sulfonated polymer, copolymer, or oligomer.

38. A method according to claim 19 wherein the combination includes HPMCAM and one or more sulfated polymer, copolymer, or oligomer.

39. A method according to claim 19 wherein the combination includes HPMCAM and acrylic based polymers, copolymers, or oligomers.

40. A method according to claim 19 wherein the combination includes HPMCAM and MVE/MA.

41. A method according to claim 19 wherein the combination includes HPMCAM and a derivative of CAP in which hydroxyl groups on the cellulose backbone of CAP have been further substituted with sulfate or sulfonate bearing moieties.

42. A method according to claim 19 wherein the combination includes HPMCAM and a derivative of HPMCP in which hydroxyl groups on the cellulose backbone of HPMCP have been further substituted with sulfate or sulfonate bearing moieties.

43. A method according to claim 19 wherein the combination includes HPMCAM and cationic polymers, copolymers, or oligomers.

44. A pharmaceutical composition for treating or decreasing the frequency of transmission of a virus selected from the group consisting of human immunodeficiency virus and herpes virus, or for preventing, or decreasing the frequency of the transmission of or for treating a sexually transmitted bacterial infection comprising an effective anti-human immunodeficiency virus amount or anti-herpesevirus amount or an effective anti-bacterial amount of, or an anti-fungal amount of a composition wherein one or more compound of Formula I is formulated together with one or more water-soluble hydrocolloids and a solublizing or emulsifying agent.

45. A pharmaceutical composition according to claim 44 wherein the compounds of Formula I includes HPMCT.

46. A pharmaceutical composition of claim 44 wherein the compounds of Formula I includes HPMCT and the concentration of said compound is present in a suitable dose that will range from about 0.001 to 25% wt/vol, preferably in the range of 0.01 to 3% wt/vol of formulated material.

47. A pharmaceutical composition according to claim 44 wherein the compounds of Formula I includes HPMCAM.

48. A pharmaceutical composition of claim 44 wherein the compounds of Formula I includes HPMCAM and the concentration of said compound is present in a suitable dose that will range from about 0.001 to 25% wt/vol, preferably in the range of 0.01 to 3% wt/vol of formulated material.

49. A pharmaceutical composition according to claim 44 wherein the compounds of Formula I includes CAT.

50. A pharmaceutical composition of claim 44 wherein the compounds of Formula I includes CAT and the concentration of said compound is present in a suitable dose that will range from about 0.001 to 25% wt/vol, preferably in the range of 0.01 to 3% wt/vol of formulated material.

51. A pharmaceutical composition according to claim 44 wherein the compounds of Formula I include a derivative of CAP wherein hydroxyl groups of CAP have been further substituted with sulfate or sulfonate bearing moieties.

52. A pharmaceutical composition of claim 51 wherein a sulfate or sulfonate modified CAP is included in the compounds of Formula I and in general is present in a suitable dose that will range from about 0.001 to 25% wt/vol, preferably in the range of 0.01 to 3% wt/vol of formulated material.

53. A pharmaceutical composition according to claim 44 wherein the compounds of Formula I include a derivative of HPMCP wherein hydroxyl groups of HPMCP have been further substituted with sulfate or sulfonate bearing moieties.

54. A pharmaceutical composition of claim 53 wherein a sulfate or sulfonate modified HPMCP is included in the compounds of Formula I and in general is present in a suitable dose that will range from about 0.001 to 25% wt/vol, preferably in the range of 0.01 to 3% wt/vol of formulated material.

55. A pharmaceutical composition according to claim 44 wherein the compound or compounds according to Formula I are used in combination with other anti-infective or spermicidal agent(s).

56. A method according to claim 1 wherein the virus is one or more members of the retrovirus family including HIV-1.

57. A method according to claim 1 wherein the virus is one or more members of the herpesvirus family including HSV2 and HSV1.

58. A method according to claim 1, wherein the therapeutic agent is administered topically.

59. The method according to claim 1 wherein the bacteria is selected from the group consisting of Trichomonas vaginalis, Neisseris gonorrhoeae Haemopholus ducreyi, or Chlamydia trachomatis, Gardnerella vaginalis, Mycoplasma hominis, Mycoplasma capricolum, Mobiluncus curtisii, Prevotella corporis, Calymmatobacterium granulomatis, and Treponema palliduin. Pseudomonas aeruginosa, Streptococcus gordonii, or S. oralis for dental plaque, Actinomyces spp, and Veillonella spp.

60. A composition of claim 44 wherein said water-soluble hydrocolloid is cationic

61. A composition of claim 44 wherein said solublizer includes glycerin.

62. A composition of claim 44 wherein said solublizer includes propylene glycol.

63. A composition of claim 44 wherein said solublizer includes a polyethylene glycol.

64. A method according to claim 1 of administering to the host a therapeutically effective amount of at least one compound according to Formula I and at least one further antiviral, antifungal, or antibacterial agent.

65. A method according to claim 2 of administering to the host a therapeutically effective amount of at least one compound according to Formula I and at least one further antiviral, antifungal, or antibacterial agent.

66. A method according to claim 7 of administering to the host a therapeutically effective amount of at least one compound according to Formula I and at least one further antiviral, antifungal, or antibacterial agent.

67. A method according to claim 11 of administering to the host a therapeutically effective amount of at least one compound according to Formula I and at least one further antiviral, antifungal, or antibacterial agent.

68. A method according to claim 15 of administering to the host a therapeutically effective amount of at least one compound according to Formula I and at least one further antiviral, antifungal, or antibacterial agent.

69. A pharmaceutical composition according to claim 44 in which the therapeutic agent can be delivered in a liquid or solid dosage form and can be incorporated into barrier devices such as condoms, diaphragms, or cervical caps, to help prevent the transmission of STDs.

70. A method for treating, or decreasing the frequency of transmission of a virus, or bacterial, or fungal infection in a host comprising administering to the host a therapeutically effective amount of at least one compound according to Formula II either alone or in combination with a pharmaceutically acceptable carrier, emulsifier, salt, or diluent, or other pharmaceutically active agent:
Wherein:
The addition of R to the oligomer, polymer, or copolymer backbone results in a new compound that is soluble and mostly dissociated in an aqueous solution over a wide range of pH (preferably from 14 to below 3.5).

71. A method for treating, or decreasing the frequency of transmission of a virus, or bacterial, or fungal infection in a host comprising administering to the host a therapeutically effective amount of at least one compound according to Formula II either alone or in combination with a pharmaceutically acceptable carrier, emulsifier, salt, or diluent, or other pharmaceutically active agent:
Wherein:
The polymer, copolymer, or oligomer backbone in Formula II can be substituted where the substituting agent R is —H, or —CH₂CH(OH)CH₃, or acetic acid, or any monocarboxylic acid, or it can be derived from trimellitic acid, or hydroypropyl trimellitic acid, or alternatively, R can be derived from any multi-carboxylic acid as shown in (but not limited to) Table 1 such that the resultant molecule will be soluble and mostly dissociated in an aqueous solution over a wide range of pH (preferably from 14 to below 3.5).

72. A method according to claim 71 wherein the therapeutic agent is the copolymer of methyl vinyl ether and maleic acid,

73. A method according to claim 72 wherein the overall molecular weight of the molecule ranges from 500 daltons to >1.5 MM daltons.

74. A method according to claim 71 wherein the therapeutic agent is the copolymer of methyl vinyl ether and maleic acid in combination with any other antiviral or antibacterial or anti-fungal agent.

75. A pharmaceutical composition for treating or decreasing the frequency of transmission of a virus selected from the group consisting of human immunodeficiency virus and herpes virus, or for preventing, or decreasing the frequency of the transmission of or for treating a sexually transmitted bacterial, or fungal infection comprising an effective anti-human immunodeficiency virus amount or anti-herpesevirus amount or an effective anti-bacterial amount of, or an anti-fungal amount of a composition wherein the copolymer of methyl vinyl ether and maleic acid is formulated together with one or more water-soluble hydrocolloids and a solublizing or emulsifying agent.

76. A composition according to claim 75 wherein the concentration of said copolymer in general is present in a suitable dose that will range from about 0.001 to 25% wt/vol, preferably in the range of 0.01 to 3% wt/vol of formulated material.

77. A method according to claim 75 for treating or decreasing the frequency of transmission of a virus, bacterial or fungal infection comprising comprising an effective anti-virus, anti-fungal or anti-bacterial amount of the pharmaceutical composition.

78. The method according to claim 75 wherein the therapeutic agent is administered topically.

79. A composition of claim 75 wherein said water-soluble hydrocolloid is cationic

80. A composition of claim 75 wherein said solublizer includes glycerin.

81. A composition of claim 75 wherein said solublizer includes propylene glycol.

82. A composition of claim 75 wherein said solublizer includes a polyethylene glycol.

83. A pharmaceutical composition according to claim 75 further includes one or more pharmaceutically acceptable carrier or excipient.
